(19) United States
(12) Patent Application Publication
(10) Pub. No.: US 2010/0159512 A1
(43) Pub. Date: Jun. 24, 2010

(54) METHOD FOR THE PREPARATION OF RECOMBINANT HUMAN THROMBIN AND FIBRINOGEN

(76) Inventor: Kurt Osther, Scottsdale, AZ (US)

Correspondence Address: THORPE NORTH & WESTERN, LLP., P.O. Box 1219, SANDY, UT, 84091-1219, US

(21) Appl. No.: 12/281,877

(22) PCT Filed: Mar. 6, 2007
(86) PCT No.: PCT/US07/05845

§ 371 Date: Jan. 26, 2009

Related U.S. Application Data
(60) Provisional application No. 60/779,474, filed on Mar. 6, 2006.
(60) Provisional application No. 60/843,945, filed on Sep. 12, 2006.

(51) Int. Cl. C12P 21/00; C12N 5/071

(52) U.S. Cl. 435/69.1; 435/366

(57) ABSTRACT

The present invention discloses a novel method for the preparation of recombinant human proteins expressed in human cells. Specifically, the present invention relates to novel methods for the preparation of human recombinant thrombin and human recombinant fibrinogen. Moreover, the method employs serum-free culturing conditions and therefore provides recombinant human proteins expressed in human cells of increased safety to the patient when used in human medical treatments. In addition, the immunogenic response to the recombinant human proteins expressed in human cells may be lower. Human recombinant thrombin is expressed in the human embryonic kidney 293 cell line and the protein can be prepared using two different routes, one starting from a point mutated prothrombin with gla and Kringle 1 and 2 domains, and maintaining these domains during the process; the other one starting with prothrombin (non-mutated), via a prethrombin with a HPC4-Kringle 2 domain and subjecting this prethrombin to a point mutation. Human recombinant fibrinogen is expressed in the human embryonic kidney 293 cell line.

This patent application hereby incorporates by reference in its entirety the material contained on the compact disc submitted to the USPTO in accordance with 37 C.F.R. §1.77(b)(4). The CD containing said material was created on Mar. 6, 2007, has a file size of 24.2 KB, and a file name of “070306 Sequence listing_WorkFile.txt”.

### FIELD OF THE INVENTION

The present invention relates to a novel concept and method for preparing compositions of recombinant human proteins, each prepared in a novel manner that create authentic or natural human proteins produced in human cells, human cell lines, human stem cells, or human precursor cells. Specifically, the present invention relates to novel methods for the preparation of human recombinant thrombin and human recombinant fibrinogen. Human recombinant thrombin can be prepared using two different routes, one starting from a point mutated prothrombin with gla and Kringle 1 and 2 domains, and maintaining these domains during the process; the other one starting with prothrombin (non-mutated), via a prethrombin with a HPC4-Kringle 2 domain and subjecting this prethrombin to a point mutation.

### TECHNOLOGICAL BACKGROUND

Thrombin and fibrinogen are proteins that are part of the fibrin clotting cascade. Thrombin is defined as a two chain, disulfide-bonded, glycosylated polypeptide that cleaves specific bonds in fibrinogen to produce fibrin monomers that self-assemble to form a fibrin clot.

When mixing the thrombin and the fibrinogen together, possibly with the addition of the bovine aprotinin, a final stage of the natural fibrin clotting cascade takes place. In addition to providing hemostasis, this fibrin sealant also provides ideal environment for fibroblastic proliferation as well as proliferation of other mesenchymal cells. The tissue sealant such as for instance Tisseel®, and modification of the individual amount of fibrinogen, thrombin and aprotinin decides the speed by which the clotting is taking place and the density of the clot. The tissue sealant combined in the manner that Tisseel is used, was tested by Mats Brittberg et al., in his dissertation from 1994, where he showed that for instance chondrocytes did in his studies not infiltrate the fibrin clot obtained using Tisseel, but the cells were merely lining up against the clot, which again might be theorized as appearing as a blocking mechanism, which would entrap chondrocytes in for instance cartilage defects, but not allowing the cells to infiltrate into the fibrin clot, also called fibrin glue or tissue sealant. Tisseel is routinely used as tissue sealant when autologous chondrocyte implantation are performed on patients, when either periosteal grafting are used as cover over the implanted chondrocytes or collagen type I/III (ChandraGide®) membranes as cover. The edge of the cover is sutured to the surrounding healthy cartilage, and tissue sealant (also called fibrin glue) such as for instance Tisseel is added to the suturing of the cover, so that cells can be injected below the cover and sealed off inside the defect. However, Mats Brittberg also found that autologous fibrin clot, made from the spontaneous clotting of the blood in the cartilage defects in the animals appeared to allow the chondrocytes to infiltrate and, contrary to Tisseel, be cultured in the fibrin clot. Recently it has been described that Tisseel® is used in a method called Matrix Assisted Chondrocyte Implantation (Marlovits S, et al. Knee Sure Sports Traumatal Arthrosc. (2005) 13:451-7; Bartlett W, et al. J Bone Joint Surg Br. (2005) 87:640-5). Tissue sealant has also been used in animals to study a possible enforcement of a myocardial infarction as a scaffold capable of preserving the myocardial wall (Christman K L, et al. Tissue Eng. (2004) 10:403).

Fibrinogen and thrombin combined components, in general, appear to be significantly better for the use as sealant of tissue, such as for instance in controlling bleeding on the cut surface of organs difficult to place sutures in organs such as kidneys, where Tisseel, when compared to CoSeal (Baxter), which is made from polyethylene glycol components, showed superior advantages in the form of clotting and adhering to surfaces, where CoSeal appeared to be significantly inferior. CoSeal could not adhere or prevent sieving of blood from a cut surface, which make it non-useable when compared to fibrinogen-thrombin combinations of tissue sealant (Bernie J E, et al. J. Endourol. (2005) 19:1122-6). FLOSEAL (Baxter), which consists of collagen particles and thrombin appearing together with Tisseel, fibrinogen and thrombin, in combination was capable of preventing more extensively bleeding areas such as tested on kidneys by L′Esparance et at (L′Esparance J O, et al. J. Endourol. (2005)19:1114-21), who combined the sealing using both types of tissue sealants, when compared to using Tisseel alone. In a number of other surgical interventions, tissue sealant, such as Tisseel has proven to be significantly better than suturing or stapling (Langrehr J M, Rozhl Chir. (2005) 84:399-402)

The ideal matrix for wound healing, hemostatic, has to be capable of adhering to any mammal tissue surfaces including bleeding surfaces to prevent bleeding during surgery and to conserve the patient's own blood volume, or at least minimize the necessity for the usage of natural blood products on the patient.

At the same time the tissue sealant having the above capabilities shall be safe in regards to minimize transmitting of pathogenic microbials and pathogenic prions including transfer of Mad Cow Disease. The current commercially available products used in large amount are tissue sealants such as Tisseel® (Baxter Immuno) and Beriplast® (Aventis/Behring); both these tissue sealants and many other tissue sealants build on the usage of natural human thrombin and natural human fibrinogen, —and furthermore, in the Tisseel—as well as in the Beriplast products the usage of bovine aprotinin; other tissue sealants available on the market are even more extensively based on bovine products.

Most of the commercial products used extensively, such as for instance Tisseel fibrin sealant (Baxter, Immuno) and alike contains bovine proteins such as aprotinin.

The Tisseel matrix is postulated to provide the optimum environment for fibroblast infiltration and proliferation, in addition providing hemostasis, tissue synthesis and wound healing as hypothesized. The natural thrombin used in Tisseel and in other tissue sealants has been used clinically in several years to control bleeding during surgery, for burns and in certain trauma situations (Nakamura at al. The Amer. Surgeon (1991) 57:226-230; Thompson et al. Opthalmology (1986) 93:279-282; Harris at al., J. Bone Joint Surg. Am. (1978) 60:454-456; Craig and Asher, Spine (1997) 2:313-317; Prasad et al.; Burns (1991) 17:70-71). Bovine thrombin is also a component of some commercial tissue sealants, also called tissue glues such as Costasis® (Angiotech Pharmaceuticals, Inc. Vancouver, BC, Canada), which also contains bovine collagen.

Commercial thrombin therapeutics are purified from pooled human and animal blood products and as such run the risk of contamination with viruses such as the HIV and hepatitis viruses. In comparing three commercial thrombin preparations, Suzuki and Sakuragawa (Suzuki and Sakuragawa, Thromb. Res. (1989) 53:271-278) found that the preparations contained contaminating proteins, and the human preparation contained immunoglobulin G, hepatitis B surface antigen antibodies and human immunodeficiency antibodies. Xenogeneic immunization with bovine thrombin has been reported in patients who have developed self-reactive antibodies to both human thrombin and human factor V (factor V is a contaminant in the bovine thrombin preparation) (Stricker et al., Blood (1988) 72:1375-1380); Flaherty and Weiner, Blood (1989) 73: 1388); Flaherty et al., Ann Int. Med. (1989) 111:631-634); Zehnder and Leung, Blood (1990) 76:2011-2016); Lawson et al., Blood (1990) 76:2249-2257); Stricker et al., Blood (1988) 72:1375-1380); Berguer et al., J. Trauma (1991) 31:408-411). In 139 patients treated with Floseal (Baxter), 25 out of 139 (18%) of the patients presented with an increased titer over baseline for bovine thrombin antibodies. Whereas in a control group, consisting of patients treated with a gelatine-containing bovine thrombin sponge, 26/131 (20%) of the patients presented with an increased titer over baseline for bovine thrombin. The corresponding numbers for bovine Factor Va were 39/139 (28%) and 43/131 (33%) for the Floseal (Baxter and Control groups, respectively. The differences in the frequency of developing either bovine thrombin or bovine Factor Va antibodies between the 2 groups were not statistically significant (Winterbottom, N. et al., J. Applied Res. (2002) Vol. 2, Number 1).

Results recently published by Wai Y et al. (Wal Y, Tsui V, Peng Z, Richardson R, Oreopoulos D, Tarlo S M, Clin. Exp. Allergy. (2003) 33:1730) indicate the potential for sensitization and clinical allergic responses to bovine thrombin when used for haemostasis topically in patients in hemodialysis and suggest that other haemostatic methods should be considered.

Bovine thrombin is used as an aid to hemostasis in medical and surgical procedures. At least 500,000 Americans are exposed to this therapeutic annually and reports suggest that exposure is associated with the development of autoreactive antibodies. To determine whether bovine thrombin can induce pathological autoimmunity Schoenecker et al. exposed non-autoimmune-prone galactose-α1-3-galactose-deficient mice to the two bovine thrombin preparations currently approved for use in the United States, and found that, like humans exposed to bovine thrombin, mice developed an immune response against the therapeutic and the xenogeneic carbohydrate galactose-α 1-3-galactose, and some mice developed autoantibodies against clotting factors. Further, unexpectedly, a single exposure to this therapeutic also induced autoimmunity in mice with features characteristic of systemic lupus erythematosus including antibodies against nuclear antigens, native DNA, double-stranded DNA, and cardiolipin. High levels of these autoantibodies were correlated with glomerulonephritis in all mice evaluated. This autoimmune syndrome was detected in mice 15 weeks after a secondary exposure to bovine thrombin and female mice were found to develop the syndrome at a significantly greater frequency than males. Thus, these studies indicate that exposure to bovine thrombin preparations can induce a pathological systemic autoimmune syndrome with lupus-like serology (Schoenecker J G, Johnson R K, Lesher A P, Day J D, et al., American J. of Pathology, (2001) 159:1957).

In addition, concerns have recently been raised regarding the possible contamination of bovine products with pathogens such as the bovine spongiform encephalitis (BSE) agent, which is not detectable or inactivatable by conventional means. Therapeutic human blood products are also subject to contamination by viral particles such as the hepatitis virus and the human immunodeficiency virus.

In other expression systems such as bacteria (e.g., E. coli), yeast systems, to which a DNA sequence is transmitted or in insect cells, avian cells, and other animal cells to which a DNA sequence is transfected, the post translation of many human proteins, especially of the types described above, such as fibrinogen, thrombin, collagens, and other cross-linking proteins such as recombinant factor XIII, which is actually, as described in U.S. Pat. No. 6,780,411 is made in transmitted yeast, —actually expressed cytoplasmically by yeast (natural fibrinogen purified from blood contains recombinant factor XIII as the cross-linking protein stabilizing the coagulation process) referring to the U.S. patents, mainly described in U.S. Pat. No. 6,780,411, does not completely resemble the authentic natural protein as much as the recombinant human protein (e.g., fibrinogen, prothrombin, construction of Prothrombin Expression Units and Expression in Mammalian Cells, thrombin, factor XIII, collagen(s) produced in a system consisting of transfected human embryonic kidney cells and other cells (e.g., HEK 293, HEK 293T, HEK 293S, HEK 293 EBNA) maintained and producing the said protein or proteins in a serum free synthetic cell culture medium system.

As described in U.S. Pat. No. 6,780,411 recombinant human Fibrinogen, for example as produced in the milk of transgenic sheep, appears to be under-sialylated, which will be a significant difference, when compared to the invention described in this patent application, where the post translation provides a more authentical natural protein, also in the case of human recombinant fibrinogen, which is a significant difference from those products, for instance produced in cloned animals such as for instance a sheep. First, since the solubility of fibrinogen is known to depend on adequate sialyation, and as fibrinogen is in any event among the least soluble of plasma proteins, there is the real possibility that not enough soluble fibrinogen would be available in the present invention to form a fibrin gel of adequate tensile strength, when compared to the fibrinogen made using the methods described in this invention. On the contrary, in this invention the fibrinogen will be co-expressed by the fibrinogen human genes encoding α, β, and γ chains.

While recombinant thrombin may be produced in a variety of hosts, the most preferred recombinant thrombin until this invention was the production in CHO cells which actually, contrary to this invention requires the presence of foetal bovine serum, and thereby adding the possible disadvantages to the proteins made first of all from a source like the CHO cells, and next relying on the addition of foetal bovine serum as a significant contaminant both in regards to non human proteins added and in regards to the inherent danger of the presence of pathogenic prions derived from diseases such as mad cow disease.

Unlike the prothrombin produced according to this present invention, other recombinant prothrombin molecules have been prepared through recombinant means, where approximately 14% of the protein was abnormally carboxylated. According to U.S. Pat. No. 5,502,034, the prothrombin made according to this invention was prepared in suitable yeast vectors for use in the present invention include YRp7 (Struhl et al., Proc. Natl. Acad, Sci. USA 76:1035-1039 (1978)), YEp13 (Broach et al., Gene 8: 121-133 (1979)), POT vectors (Kawasaki et al, U.S. Pat. No. 4,931,373, which is incorporated by reference herein), pJDB249 and pJDB219 (Beggs, Nature 275:104-108 (1978)) and derivatives thereof. The prothrombin made in yeast will not be identical to authentic natural human thrombin.

In U.S. Pat. No. 6,037,457, a recombinant fibrinogen is produced in a preferred embodiment of the invention Chinese Hamster Ovary (CHO) cells which were used to produce recombinant fibrinogen. Illustrative conditions under which mammalian cells can be cultured to express fibrinogen are as follows: cells are grown in roller bottles with adherent microcarrier beads in a total of 200 ml of serum free medium (Dulbecco's Modified Eagle's Medium/F12 medium containing 10 IU penicillin/ml, 10 mg streptomycin/ml, 10 U aprotinin/ml, and 10 .mu.g/ml each of insulin, sodium selenite, and transferrin) at 37.degree. C. for three weeks prior to commencing the collection of conditioned culture medium every four to seven days. However, none of the other proteins included in this invention is made in the same type of cell system, and is certainly not done in a human cell system such as for instance human kidney cell and human retina-derived PER-C6 cells or human embryonic kidney cells, or even in stem cells as well as precursor cells. The above invention actually only deals with recombinant fibrinogen alone, and therefore, does not solve the problem around having a complete pure recombinant human post translated system without the content of serum proteins.

### SUMMARY OF THE INVENTION

The present invention provides recombinant human proteins, notably prothrombin, prethrombin, thrombin and fibrinogen, vectors and human expression systems for use in the preparation of the proteins. Moreover, the recombinant human thrombin provided in an embodiment of the invention comprises a mutation, (M84A), or in a more exact and descriptive manner according to this invention, the prothrombin analogue previously called M84A, will be “Prothrombin Analogue M400A”, and the prethrombin analogue also previously called M84A, will be “Prethrombin Analogue M256A”. The invention also relates to the use of the recombinant human proteins in medicine as described herein for the corresponding non-recombinant proteins. Two different routes for obtaining thrombin is described, one starting from a point mutated prothrombin with gla and Kringle 1 and 2 domains, and maintaining these domains during the whole process; the other one starting with prothrombin (non-mutated), via a prethrombin with a HPC4-Kringle 2 domain and subjecting this prethrombin to a point mutation.

The uniqueness of the invention described in this patent application to produce human recombinant authentic proteins builds upon using plasmid cDNA transfected human cells, capable of secreting the protein expressed by the particular message transfected. In this invention human cells such as human kidney cell, more specifically human embryonal kidney (HEK) cell lines (e.g., HEK 293, HEK 293T, HEK 293S, HEK 293 EBNA), and human retina-derived PER-C6 cells, or other cells such as stem cells or precursor cell lines are propagated in are producing the proteins in serum free synthetic medium.

The significant advantage in this novel human cell culturing system including stem cells and/or precursor cells, more specifically a kidney cell system, or even more specifically, a human embryonic cell line and other cell lines, called HEK 293, HEK 293T, HEK 293S, HEK 293 EBNA, and a human kidney cell and human retina-derived PER-C6 cells systems grown, maintained and capable of producing the proteins post translated from transfected DNA libraries into these cells under the unique serum free synthetic cell culture medium system, the combination of the above, is the central part of this new invention.

The efficacy of the tissue sealant made from the combination of these human recombinant proteins is, in efficacy, comparable to other known tissue sealants such as for instance Tisseel® (Baxter Immuno, Vienna, Austria), but the significant difference between the current tissue sealant available on the market—compared to the tissue sealants described in this invention, called HUMA-SEALANTT™ and HUMA-SEALANT™-C is the fact that in the current tissue sealants on the market, some of the proteins used, are of natural origin and are made from human donor blood and/or some of the proteins are made from bovine material, making these products significantly unsafe when compared to HUMA-SEALANT™ and HUMA-SEALANT™-C , in regards to transfer of infections such as hepatitis, the issue around bovine proteins transfer of abnormal prions or transfer of Mad Cow Disease to patients; furthermore the bovine (xenogeneic) proteins will produce immunity in patients adding additional dimensions to the possible side effects of the current tissue sealants available on the market; on the contrary, the HUMA-SEALANT™ tissue sealant system, which this invention among others concerns, consists of recombinant human proteins post-translated in transfected human cells to obtain authentic, natural proteins making this tissue sealant system optimal in regards to safety both what concerns infectivity and immunogenicity.

The efficacy of the tissue sealant made from the combination of the human recombinant proteins claimed in this patent application is comparable to or better than other known tissue sealants made from natural protein sources such as blood, plasma or other body fluid from humans and from animals such as bovine species used in fibrin glues or tissue sealants such as for instance Tisseel® (Baxter Immuno, Vienna, Austria). One of the reasons why for instance one type of the recombinant proteins, namely the recombinant human thrombin mutants described in this patent application has intentionally been selected from the point of view that these mutants appear to show optimal ability to cleave fibrinogen and recombinant fibrinogen and at the same time show low efficacy in cleaving protein C, which is significantly different from natural thrombin. The significant difference between the current tissue sealant available on the market—compared to the tissue sealants described in this invention, named HUMA-SEALANT™ and HUMA-SEALANTT™-C, is—that in the current tissue sealants on the market, the substantial proteins used, are of natural origin and are made from human donor blood and/or some of the proteins are made from bovine material, making these products significantly unsafe when compared to HUMA-SEALANTT™ and HUMA-SEALANTT™-C , in regards to transfer of infections such as hepatitis, the issue around bovine proteins transfer of abnormal prions or transfer of Mad Cow Disease (BSE) causing prions to patients; furthermore the bovine (xenogeneic) proteins will produce immunity in patients adding additional dimensions to the possible side effects of the current tissue sealants available on the market; on the contrary, the HUMA-SEALANT™ tissue sealant system, presented in this invention, consists of recombinant human proteins post-translated in transfected human cells to obtain authentic, natural proteins with optimal activity and with a high degree of lot to lot reproducibility, the method of which also make this tissue sealant system optimal in regards to safety both what concerns infectivity and immunogenicity. The novel and unique processing of human safe recombinant proteins for instance used to produce the tissue sealant products described in this invention is therefore significantly different when compared to the current tissue sealants available for use in patients and, furthermore, is significantly distant to other tissue sealants described in available patent applications and accessible patents.

The ideal matrix for wound healing, sealant, hemostatics, etc. has to be capable of adhering to any mammal tissue surfaces including bleeding surfaces to prevent bleeding during surgery and to conserve the patient's own blood volume, or at least minimize the necessity for the usage of natural blood products on the patient. At the same time the tissue sealant having the above capabilities shall be safe in regards to minimize transmitting of pathogenic microbials and parthogenic prions including transfer of Mad Cow Disease (or BSE). The current commercially available products used in large amount are tissue sealants such as Tisseel® (Baxter Immuno) and Beriplast® (Aventis/Behring); both these tissue sealants and many other tissue sealants build upon the usage of natural human thrombin and natural human fibrinogen, —and furthermore, in the Tisseel—as well as in the Beriplast products the usage of bovine aprotinin; other tissue sealants available on the market are even more extensively based on bovine products.

The novel and unique processing of safe recombinant proteins for instance used to produce the tissue sealant products described in this invention is therefore significantly different when compared to the current tissue sealants available for use in patients and furthermore is significantly distant to other tissue sealants described in available patent applications and accessible patents.

In particular, this particular invention is focusing on models capable of acting as Tissue Sealants and hemostatics as well as carriers of cells and carriers of drugs. The present invention is applying protein informatics to accurately identify proteins and isozymes or isoforms from the human genome, identifying the gene or genes in question, and using a novel vector system the gene or genes in question is transfected into a well defined human cell line, a stem cell line, a precursor cell line, or more specifically a human kidney cell and human retina-derived PER-C6 cells and human retina-derived PER-C6 cells, and even more specifically into a human kidney cell and human retina-derived PER-C6 cells or a human embryonal kidney cell line and other cell lines called HEK 293, HEK 293T, HEK 293S, HEK 293 EBNA. This system is in this invention used for producing proteins such as recombinant human thrombin, recombinant human fibrinogen and recombinant human collagen, or more specifically recombinant human collagen of one of the around 19-20 types of human collagens types I, II, III, IV, V, VI, VII, VIII, IX, X, XI, XII, XIII, XIV, XV, XVI, XVII, XVIII, and XIX as well as other known human collagens.

One aspect of the present invention is to provide methods for producing thrombin using recombinant methods in host cells, such as human cells, human cell lines, human stem cells, human precursor cells, more specifically, a human kidney cell and human retina-derived PER-C6 cells line, and even more specifically, a human embryonal kidney cell line and other cell lines (e.g., HEK 293, HEK 293T, HEK 293S, HEK 293 EBNA).

### DETAILED DESCRIPTION OF THE INVENTION

One objective of the invention is among others to provide biological and very safe tissue sealants, made in a novel way, in which, contrary to other compositions of tissue sealant, exclusively consists of producing all the interactive proteins each in human cell lines, which has been transfected with cDNA copies of the proteins claimed in this invention, and which is propagated by processing the cells containing the copy or copies of cDNA in serum-free medium and thereafter harvesting the specific proteins produced by said cells in serum-free medium to approach similarity to authentic human selected proteins from the cells in serum-free medium.

The present invention relates to a novel concept and method for combining certain recombinant human thrombin with fibrinogen as such as well as certain human recombinant fibrinogens to create a stable product, with an optimal fibrinogen cleavage activity and with a low effect on activation of protein C.

It is within the scope of this invention to include mammal cells and specifically the cells claimed in U.S. provisional patent application 60/779,474, which is incorporated herein by reference, with incorporated cDNA, aimed at expressing prethrombin, which are cultured according to proprietary methods in a serum-free synthetic culture medium containing nutrients required for the growth of the host cells and at the same time the secretion of the post translated prethrombin.

All the other methods described by the use of human DNA library from which cDNA libraries are produced by inserting into an expression vector driven by a strong promoter, described under “Construction of the expression vector” can be used. A stable human cell will under then produce said proteins, in certain individual amounts of each of said human recombinant proteins from one or more of the cell tines, or some of the recombinant proteins, and will create a fast working end product consisting of proteins involved in the controllable gelling or coagulation, and will be free of contaminating proteins for instance derived from any form of use of mammal cells as described in U.S. Pat. Nos. 6,780,411, 5,476,777, 5,502,034 and 5,572,692, where it, on the contrary, is described that when using a transfecting method for instance recombinant human thrombin is produced either in inactive form and activated as part of the purification process. Processes for the production of rhThrombin in this way are disclosed in U.S. Pat. Nos. 5,476,777, 5,502,034 and 5,572,692, the teachings of which are incorporated herein by reference. In all these cases described, where mammal cells or other cells are used for obtaining either prothrombin such as gal prothrombin, where it is stated that the cells used to produce these proteins and actually also recombinant human proteins such as for instance recombinant human fibrinogen, as well as cross linking recombinant human proteins such as recombinant human factor XIII or other cross linking recombinant human proteins such as collagens and in particular recombinant human collagen type I and/or recombinant human collagen type III.

Any other protein combinations used in the field of creating tissue sealant for human use that are used to produce an optimal combination of proteins with the highest purity and the least risk for contaminations can not be compared to the highest purity, the smallest chance for contamination of infections, the highest level of compatibility of said proteins can not be obtained with hitherto available or accessible products, due to the fact that except for the human sealant product made as described in this invention, namely produced by transfected human cells or in particular human kidney cell and human retina-derived PER-C6 cells devoid of any added proteins usually added to the cell culture, cell growth or protein producing cell medium often consisting of natural proteins, and such as for instance bovine (e.g., foetal bovine serum) or human serum with all the uncertainties that these combination of proteins added to the cell culture might contribute, due to the fact that the only proteins that may be released from the human cells cultured, and maintained as well as brought to production of proteins in serum free synthetic medium.

All these proteins are according to this invention made in transfected human cells in serum free synthetic cell culture medium. This method will then definitely distance the proteins produced by the human cells, and more specifically human kidney cell and human retina-derived PER-C6 cells or even more specifically human embryonic kidney cells and other cell lines (e.g., HEK 293, HEK 293T, HEK 293S, HEK 293 EBNA) propagated in the serum free synthetic cell culture medium to be proteins that require authentic post-translational modification in order to have high biological activities or authentic antigenicity (for antibodies). Stem cells or precursor cells may also be used. At the same time the proteins produced in this manner will be less heterogenic, but on the contrary more homogenous, enabling a better consistency in the final protein product, more reproducible from lot to lot, functionally more active and less immunogenic due to the lesser difference in the sugar molecules, which otherwise may be obtained in cells such as insect cells or CHO cells. At the same time the final product will also be virus free. According to the method as claimed in this invention we are capable of generating stable cell lines and easily make cells adapted to serum-free suspension.

In this manner the proteins purified from the human cell culture such as for instance the human kidney cell and human retina-derived PER-C6 cell cultures in serum free synthetic cell culture medium will be novel and very different from the protein mixture from any of the transfected cell lines to which serum proteins such as for instance fetal bovine serum or other natural proteins, human or other mammal natural proteins are added and will in more or possibly less amount be present in the final “purified” protein to be harvested. It can for instance not be excluded that proteins added to the transfected mammal cell cultures, to which serum (bovine, human or other species of proteins) is still to be considered theoretically and possibly also in practice—present in the final product, which may not exclude microorganisms, such as virus, virions, pathological prions, as for instance those sometimes present in animals that might be exposed or contaminated with “mad cow disease”.

It is within the scope of this invention to include mammal cells or more specifically the human cells claimed in U.S. provisional patent application 60/779,474 with incorporated cDNA, when aimed at expressing gla-domain containing prothrombin. The N-terminal Gla domain of human prothrombin is a functional unit that has a binding site for factor Va. The prothrombin Gla domain is important for interaction of the substrate with the prothrombinase complex. The cells are cultured according to proprietary methods in a serum-free synthetic culture medium containing nutrients required for the growth of the host cells and at the same time the secretion of the post translated gla-domain containing prothrombin, which unique to this invention and which is certainly significantly different from gla-domainless prothrombin or prethrombin.

It is also within the scope of this invention to use human recombinant collagens, such as recombinant human collagen type I and/or collagen type III or a mixture of collagen type I and Ill, where it is shown in the U.S. provisional patent application No. 60/722,366 where the very small amount of thrombin used to enhance the collagen and fibrinogen gelating product, which provides an even stronger and better adhering tissue sealant.

It is anticipated that the proteins according to this invention are as near as human natural authentic proteins so that they do not induce immunogenicity in man.

The tissue sealant disclosed in this invention, made of for instance recombinant human thrombin or recombinant thrombin mutants or analogues with the previously described pro-coagulation effect (enzymatically cleaving fibrinogen or activating fibrinogen to build fibrin), which again has showed to have a low enzymatic effect against protein C, and an almost equal or nearly equal signaling PAR 1 effect can be included in the product, which is considered to be produced under one trade name, HUMA-SEALANTT™, to, among other numerous effects and abilities (e.g., a significant cell friendly product) to be used in the same manner as for instance Tisseel, —but unlike Tisseel, and as previously described, as a vehicle or carrier for cell delivery, has to be capable of adhering well to a targeted tissue for days and even weeks to enable the matrix to produce the effect needed to be efficient in all of the above capabilities without being xenogenic, allergenic, immunogenic or without carrying pathogenic microbials as well as not exhibiting any significant toxicity.

The HUMA-SEALANT™ tissue sealant consists, as many of the competitors' fibrin sealants, at least of two active components, however as recombinant mammal fibrinogen and, more specifically a recombinant human fibrinogen, where the fibrinogen within the scope of this invention is made in cells where the cDNA contains Aα, Bβ, γ fibrinogen expression thus capable of transcribing or producing a recombinant human Aα, Bβ, γ fibrinogen. The other component of the HUMA-SEALANT™, recombinant thrombin or more specifically recombinant human thrombin for instance of the types described in this invention, which also is present as natural proteins in e.g., Tisseel, will, when mixed, simulate the final stages of the natural coagulation cascade, forming a fibrin clot, or a gelation and most probably also having an adhering effect, also defined as a biomatrix, but according to this invention a recombinant biomatrix, or even more specifically, a recombinant mammal biomatrix.

### DEFINITIONS

An Expression Vector is usually a DNA molecule, which contains, among others, a DNA sequence, which is encoding a protein of interest together with a promoter and other sequences, such as a transcription terminator and polyadenylation signal, that facilitate expression of the protein.

The expression vectors contain genetic information which provides for replication in a host cell, either by autonomous replication or by integration into the host genome. It is evident to one skilled in the art that such information that provides for the autonomous replication of an expression vector in a host cell encompasses known yeast and bacterial origins of replication.

Transformation is generally applied to microorganisms.

Transfection is generally applied to cells from multicellular organisms.

Both processes consist of stably and hereditably changing the genotype of a recipient cell by introduction of purified DNA, a part selected from a DNA library.

Cultured cell: A cell capable of being cultured in medium over a number of generations. In the case of cells derived from multicellular organisms, a cultured cell is a cell isolated from the organism as a single cell, a tissue, or an explant from a tissue.

A DNA Construct is a DNA molecule, or it could be a clone of such a molecule, and could be either single or double-stranded. This molecule will have been modified through human manipulation of certain segments of DNA combined and juxtaposed in a manner, which normally does not exist in nature. A DNA construct may contain operably linked elements, which direct the transcription and translation of the DNA sequence encoding the polypeptides that are of interest. Such elements include promoters, enhancers and transcription terminators. If such a DNA construct encompass the encoding of a polypeptide of interest, which contains a secretory signal sequence, the DNA construct such elements will be considered to be capable of directing the secretion of the polypeptide.

In order to easily discriminate between the individual expressions such as prothrombins (either gla-domain (containing) prothrombin, gla-domainless prothrombin) as used by Zymogenetics for their production of recombinant human thrombin, prethrombin, and prethrombin-1, FIG. 1-4 provide an overview over these individual terms. As illustrated in FIG. 4, prethrombin is prothrombin without gla domain and without Kringle I domain (Gla-less domainless prothrombin). To facilitate cleavage and/or purification HPC4 epitope was added on N-terminal. The first part of FIG. 4 shows the prothrombin including the Gla domain, and the Kringle 1 and Kringle 2.

HPC 4 is defined as an attached marker, to which one has a monoclonal antibody to identify the molecule during the process of splitting the prothrombin to prethrombin.

By the prefix “h” of proteins is intended to mean the human wild type variant of the protein. Wild type is normally prefixed by wt.

By the prefix “r” of proteins is intended to mean a recombinant protein, which has been prepared by recombinant expression.

By the prefix “rh” of proteins is intended to mean the human wild type variant of the protein which has been prepared by recombinant expression.

By the term “sequence identity”, such as “nucleic acid sequence identity” or “amino acid sequence identity” is intended to mean a quantitative measure of the degree of homology between two amino acid sequences or between two nucleic acid sequences that are optimally aligned. The two sequences must be aligned to give the best possible fit, allowing the insertion of gaps or, alternatively, truncation at the ends of the polypeptide sequences or nucleotide sequences. Sequence identity as used herein is the number of aligned amino acids which are identical, divided by the total number of amino acids in the shorter of the two sequences being compared.

By the term “more efficiently” in relation to clotting of fibrinogen by recombinant human thrombin as compared to human natural thrombin is intended to mean that a smaller amount of recombinant human thrombin is needed to provide a similar response to human natural thrombin.

Usage of filtrate through MWCO provides dialysis membrane pore sizes that are characterized by the molecular weight at which 90% of the solute will be retained (prevented from permeating) by the membrane. The permeability of a solute is dependent upon the shape of the molecule, its degree of hydration and its charge. Each of these may be influenced by the nature of the solvent, the pH and the ionic strength. As a general rule, it is best to choose a MWCO that is half the molecular weight of the solute to be retained. For effective separation, please refer to the following formula as a guide:

$$\frac{M W retained}{M W passed} \geq 25$$

This maximizes the sample recovery and minimizes the time required for dialysis.

### Protein Glycosylation

It is an important part of this invention to assure that the recombinant human proteins are glycosylated correctly. The glycosylation of recombinant proteins in general, especially those destined for potential administration to human subjects, is of much larger critical importance than previously believed. Glycosylation profoundly affects biological activity, function, clearance from circulation, and crucially, antigenicity (Brooks, S A, Molecular Biotechnology, (2004) Vol 28, 241-256(16)) This Brooks Review gives a brief overview of human N- and O-linked protein glycosylation, summarizes what is known of the glycosylation potential of the cells of nonhuman species, and presents the implications for the biotechnology industry.

The cells of non-human species do not glycosylate their proteins in the same way as human cells do. In many cases it has been found that the differences between “human” glycosylation and “animal” glycosylation are profound. Overall, it is known that the more distant, in evolutionary terms, such as bacteria, yeasts, fungi, insects, and plants, the species used most commonly in expression systems have glycosylation repertoires least like humans, and even cells originated from animals such as for instance in family with rodents, hamsters, and actually up to the animals nearest humans, the pig, there are significant differences in the glycosylation which possibly also can explain that the apparent activity of even proteins, such as for instance recombinant human thrombin in produced hamster cells show less biological activity, different function, clearance, and, again crucially, the antigenicity. Therefore, in regards to recombinant human thrombin described in the following patents: in U.S. Pat. No. 5,527,692, in which BHK 570 cells and yeast were described as selected for the recombinant thrombin production; in U.S. Pat. No. 5,502,034 in which BHK 570 cells are used for the transfection of Gla-domainless prothrombin, activated to recombinant thrombin (wild type), was claimed to react with fibrinogen, but not specifically with recombinant human fibrinogen or recombinant human authentic fibrinogen. So, the host cells used as described in these patents are of either animal origin or of yeast origin, and the glycosylation is therefore not considered equal to or anywhere near to the glycosylation obtained by transfecting the human genes to be expressed into human cells, such as human embryonic kidney cells (HEK 293) as have been used in this invention.

In U.S. Pat. No. 5,476,777 the same cell lines are used for transfection of the gla-domainless prothrombin, and among others purified using Sepharose columns and activated using snake venom. The resulting glycosylation of the final product described in this patent will be as different as what has been described in the above patents (U.S. Pat. No. 5,527,692 and U.S. Pat. No. 5,502,034).

It is within the scope of this invention to produce recombinant human authentic thrombin, due to the fact that the prothrombin was transfected in to human embryonic kidney cells in synthetic medium, and it is also within the scope of this invention to produce recombinant authentic fibrinogen, containing the 6 polypeptide chains, 2Aα, 2 Bβ, and 2 gamma chains constituting the intact authentic human fibrinogen, where we in the invention also have significantly purposely focused on ending up with the correct glycosylation as possible for human use, meaning that we avoided using animal cells, insect cells, yeast or bacteria as host for our two products, recombinant human thrombin or more specifically recombinant human thrombin analogue with a site mutation of Chy 84 from methionin to alanin, which we believe will not cause any antigenecity in humans, and the glycosylation will be correct, because the thrombin or thrombins claimed in this invention are produced using transfection of the plasmid into human cells, e.g., human embryonic kidney cells. Furthermore, the cells were cultured in serum free synthetic medium as a suspension culture, which appeared to give significantly higher yields, than previously described to be possible to achieve.

Many proteins in eukaryotic cells are glycoproteins because they contain oligosaccharide chains covalently linked to certain amino acids. Glycosylation is known to affect protein folding, localization and trafficking, protein solubility, antigenicity, biological activity and half-life, as well as cell-cell interactions.

Protein glycosylation can be divided into four main categories mainly depending on the linkage between the amino acid and the sugar. These are N-linked glycosylation, O-linked glycosylation, C-Mannosylation and GPI anchor attachments. N-glycosylation is characterized by the addition of a sugar to the amino group of an asparagine. In O-glycosylation, a sugar is attached to the hydroxyl group of a serine or threonine residue.

Complex carbohydrates are involved in multiple biological processes, from protein folding, oligomerization and stability, to the immune response and host-pathogen interactions (Varki, 1993). Glycoconjugates also play important roles in developmental processes, as revealed by the pathology of human diseases caused by abnormal glycosylation (Freeze and Aebi, 2005) and genetic studies in model organisms (Haltiwanger and Lowe, 2004).

There is a significant difference in glycosylation between different species. Human glycoproteins are glycosylated with a extremely diverse heterogeneous array of complex N- and O-linked glycans, which are the product of the coordinated activity of enzymes resident in the endoplasmic reticulum and Golgi apparatus of the cell. Glycosylation of proteins is highly regulated and changes during differentiation, development, under different physiological—and cell culture species from which the cell cultures derive, meaning that there is pronounced differences between the complex glycosylation in human cells versus animal cells, which wilt most certainly affect biological activity function, clearance in the organism, and of course antigenicity (Brooks S A, Mol. Biotechnol. (2004), Vol 28, 241-255). In many cases, the differences are profound. Overall, the species most distant to humans in evolutionary terms, such as bacteria, yeasts, fungi, insects and plants—the species used most commonly in expression systems—have glycosylation repertoires least like our own.

In addition, according to Apollo Cytokine Research (San Francisco, US) recombinant human proteins, in the case of the present invention recombinant human thrombin and recombinant human fibrinogen exhibits different glycosylation patterns when expressed in transfected human cells (e.g. HEK 293) as compared to expression, e.g., in other mammalian cells such as hamster cells (e.g., CHO, which is used by Zymogenetics for production of recombinant human thrombin) (http://www.apollocytokineresearch.com/Scientifidposters.html). The data from Apollo Cytokine Research shows that the protein TNF RII-Fc expressed in hamster cells (CHO cells) is more immunogenic, i.e. elicits immune reactions that yield production of antibodies, in mice than when expressed in human cells (modified 293).

Immunogenetic properties of proteins are determined both by their amino acid sequence and their glycosylation pattern.

### Thrombin

Host cells containing DNA constructs of the present invention are then cultured to produce prothrombin, which in this particular invention will be activated the thrombin during purification by proteases such as factor Xa. The cells with incorporated cDNA are cultured according to proprietary methods in a serum-free synthetic medium containing nutrients required for the growth of the host cells and at the same time the section of the post translated protein or polypeptide.

Thrombin is defined as a two chain, disulfide-bonded, glycosylated polypeptide that cleaves specific bonds in fibrinogen to produce fibrin monomers that self-assemble to form a fibrin clot.

Host cells containing DNA constructs of the present invention are unique because unlike the methods used by Zymogenetics and others aimed at producing prothrombin, is in this particular invention prethrombin (which is different from prothrombin) will be activated to thrombin.

Host cells containing DNA constructs of the present invention are unique because unlike the Gla-domainless prothrombin methods used by Zymogenetics and others aimed at producing prothrombin with gla-domainless prothrombin-, is in this particular invention gla-domain-prothrombin (which is completely and significantly different from gla-domainless prothrombin) will be activated to thrombin. The presence of the gla-domain can be evidenced by using a monoclonal antibody that specifically recognize gamma-carboxyglutamyl (Gla) residues in proteins such as gla-domain containing prothrombin using a method consisting of monoclonal antibodies specific for Gla residues, as described by Brown et al. (Brown M A, Stenberg L M, Persson U, Stenflo J, J. Biol. Chem. (2000), 275:19795-19802).

The recombinant human thrombin mutants, that specifically have proven to be procoagulant thrombin mutants (with high procoagulation effect) and with the same signalling enzyme effects as the Wild Type of thrombin on cells, such as for instance mesenchymal cells. Of the procoagulant thrombin mutants which also have PAR1 signal enzyme effect on cells when compared and low anticoagulant effect—either as low as wild type thrombin or even lower. Among those recombinant thrombin mutants are for instance the type called W215P.

Of other recombinant human thrombin mutants that are within the scope of this invention, are such types as M84A, also called “Thrombin Analogue M400A” (when made from prothrombin analogue M400A), and “Thrombin Analogue M256A (when made from prethrombin analogue M256A), which clots fibrinogen at a faster rate than the wild type and the arg77A (or R77aA) mutant that lacks autolytic activity. Using the above nomenclature for Thrombin analogue M400A, when made from prothrombin analogue M400A, and Thrombin analogue M256A, when made from prethrombin analogue M256A, one can always easily distinguish, by which method the thrombin analogue in question was made, instead of having M84A as the common denominator for both activated thrombins. In the case of R77aA, the prothrombin analogue would be “Prothrombin analogue R393aA”, and the R77aA analogue from prethrombin would be “Prethrombin Analogue R249aA”.

Also within the scope of this invention, recombinant human thrombin mutants showing low grade—or no autolysis may be selected among the types of recombinant thrombin mutants as for instance arg77A (or R77aA). The specific aim is to produce a recombinant thrombin mutant, which include at least one or more of the recombinant human thrombin mutants described above, that can be stored refrigerated and still retain their enzymatic activity to polymerize fibrinogen and especially recombinant human fibrinogen, for instance made in mammal cells.

One object of the present invention is to provide methods for producing thrombin using recombinant methods in host cells to primarily produce precursor to thrombin called prethrombin, —such as human cells, human cell tines, human stem cells, human precursor cells, more specifically, a human kidney cell and human retina-derived PER-C6 cells line, and even more specifically, a human embryonic kidney (HEK) cell lines (e.g., HEK 293, HEK 293T, HEK 293S, HEK 293 EBNA).

One object of the present invention is to provide methods for producing thrombin using recombinant methods in host cells to primarily produce precursor to thrombin called gla-domaine containing prothrombin, —such as human cells, human cell lines, human stem cells, human precursor cells, more specifically, a human kidney cell and human retina-derived PER-C6 cells line, and even more specifically, a human embryonic kidney (HEK) cell lines (e.g., HEK 293, HEK 293T, HEK 293S, HEK 293 EBNA).

These recombinant human thrombin types will be compared to wild type human thrombin and will be recombinant human thrombin analogues or mutants.

The thrombin mutants that are part of this invention that retain comparable fibrinogen cleavage activity, but have higher expression level may have the advantage of being produced at lower manufacturing cost.

It is also within the scope of this invention that thrombin mutants retain comparable expression level but display higher fibrinogen cleavage activity and thus these mutants may lower therapeutic dosage.

Further, it is within the scope of this invention that the thrombin mutants that display reduced autolysis, which are an integrated part of this invention, may facilitate purification process and storage.

As described above it is further envisioned in this invention that mutants that lack protein C activation may have improved coagulation activity.

Mutation can be identified by sequence comparison among thrombin family members, rational site-directed mutagenesis or alanine-scanning. It is envisioned genes can be modified via insertions, substitutions, deletions or domain swapping.

The methods within the scope of this invention is built on producing recombinant mammal thrombin or more specifically recombinant human thrombin and even more specific recombinant human thrombin analogue(s), and yet even more specific a recombinant human thrombin analogue, called M84A; when all of the above recombinant human thrombins or thrombin analogue(s) are made from “gla domain prothrombin” gene, meaning that the deduced protein of recombinant human prothrombin contains signal peptide, propeptide, Gla domain, two kringle domains and a (two-chain) protease domain, the M84A analogue would be called “thrombin analogue M400A. The Gla domain is seen in FIG. 5.

In a specific embodiment of the present invention the gene sequence expressed according to the invention is any molecule that exhibit thrombin activity.

The initial fibrinogen polymerization activity can be determined by spectrophotometry.

In a specific embodiment of the present invention the gene sequence expressed according to the invention is any sequence, which encodes a molecule that exhibit thrombin activity. The gene sequence expressed according to the invention can encode natural human thrombin or any mutant thereof. Thus, in one embodiment of the invention, prothrombin encoded by the polynucleotide has a sequence 100% identical to the human prothrombin sequence.

### Fibrinogen

Such recombinant human fibrinogen contain six polypeptide chains, 2 Aα, (blue) 2 Bβ, green and 2γ, red, in a trinodular shape with a central E nodule linked to two distal D nodules (see FIG. 12) are prepared individually in a novel manner that create authentic or natural human proteins produced in mammal cells or human cells, mammal or human cell lines, mammal or human stem cells, or mammal or human precursor cells.

Thrombin, including the recombinant human thrombin or the recombinant human thrombin analogue(s) encompassed in this invention catalyzes the conversion of fibrinogen, which has a trinodular shape by attaching to the FpA site at the central E nodule—linked to the two distal D nodules of the fibrinogen molecule to fibrin monomers. The fibrin monomers interact to form double-stranded protofibrils. The protofibrils aggregate to form fibrin fibres, and these fibrin fibres can theoretically be stabilized by factor XIIIa-catalyzed formation of crosslinks. The conversion of fibrinogen and in this case the conversion of recombinant human authentic fibrinogen can also be explained more simply as the conversion of fibrinogen to fibrin being triggered by thrombin, which cleaves fibrinopeptides A and B from alpha and beta chains, and thus exposes the N-terminal polymerization sites responsible for the formation of the soft clot.

in a specific embodiment of the present invention the gene sequence expressed according to the invention may be any sequence, which encodes a molecule that is a fibrinogen analogue, i.e. a molecule activated by cleavage by a serine protease such as thrombin and takes part in the clotting cascade. The gene sequence expressed according to the invention can encode natural human fibrinogen or any mutant thereof.

### Suitable Expression Systems

Host cells for use in practicing this present invention can primarily include a well defined human cell lines, known to secrete proteins or peptides, which closely resembles the natural counterpart of proteins or peptides.

The present invention is applying protein informatics to accurately identify proteins and isozymes or isoforms from the human genome, identifying the gene or genes in question, and using a novel vector system the gene or genes in question is transfected into a well defined mammal or human cell line, a stem cell line, a precursor cell line, or more specifically a human kidney cell and human retina-derived cells, and even more specifically into a human kidney cell and human retina-derived PER-C6 cells or human embryonic kidney cell lines (HEK) as for instance HEK 293, HEK 293T, HEK 293S, HEK 293 EBNA. This system is in this invention used for producing proteins such as recombinant human thrombin of the types described in this invention, recombinant human fibrinogen and recombinant human collagen, or more specifically recombinant human collagen of one of the around 19-20 types of human collagens types I, II, III, IV, V, VI, VII, VIII, IX, X, XI, XII, XIII, XIV, XV, XVI, XVII, XVIII, and XIX as well as other known human collagens.

### Construction of the Expression Vector

Mammalian expression vectors for use in carrying out the production of the proteins, claimed in this invention will include a promoter capable of directing the transcription of a cloned gene or cDNA. Preferred promoters include viral promoters and cellular promoters. Viral promoters could for instance be the immediate early cytomegalovirus promoter (Boshart et al., Cell (1985) 41:521-530) or the SV40 promoter (Subramani et al., Mol. Cell. Biol. (1981) 1:854-864).

The cloned DNA sequences may be introduced into cultured mammalian cells using a variety of methods, for example, calcium phosphate-mediated transfection, described by several authors is one preferred method (Wigler M, Pellicer A, Silverstein 5, Axel R. Cell (1978), 14: 725; Corsaro C M, Pearson M L, Somatic Celt Genetics (1981), 7: 603; Graham F L, van der Eb A J, Virology (1973) 52:456). Electroporation is another technique used for introducing cloned DNA sequences into mammalian cells as previously mentioned (Neumann E, Schaefer-Ridder M, Wang Y, Hofschneider P H., EMBO J. (1982) 1:841-845).

Cloned DNA sequences from DNA libraries may be introduced into cultured mammalian cells by, for example, calcium phosphate-mediated transfection (Graham F L and Van der Eb A J, Virology (1973) 52:456; Wigler M, Pellicer A, Silverstein S, Axel R., Cell (1978) 14:725; Corsaro C M and Pearson M L, Somatic Cell Genetics (1981) 7:603).

Neuman et al's technique introducing DNA sequences into mammalian cells by the use of electroporation may also be used (Neumann E, Schaefer-Ridder M, Wang Y, Hofschneider P H., EMBO J. (1982) 1:841-845), such as Electric impulses (8 kV/cm, 5 microseconds), which were found to increase greatly the uptake of DNA into mouse lyoma cells by electroporation in high electric fields.

To direct proteins of the present invention into the secretory pathway of human cell lines such as for instance human kidney cell and human retina-derived PER-C6 cells, human embryonic kidney cells (e.g., HEK 293, HEK 293T, HEK 293S, HEK 293 EBNA), stem cells, or even precursor cells, at least one signal sequence is linked to the DNA sequence of interest. Preferred signals may, among others, include the alpha factor signal sequence (pre-pro sequence; Kurjan and Herskowitz, Cell 30:933-943 (1982); Kurjan et al., U.S. Pat. No. 4,546,082; Brake, U.S. Pat. No. 4,870,008), and the PHO5 signal sequence (Beck et al., WO 86/00637), and the BAR1 secretory signal sequence (MacKay et al., U.S. Pat. No. 4,613,572; MacKay, WO 87/002670).

A selectable marker is normally used to identify cells along with the gene or cDNA of interest. Such preferred selectable markers for use in cultured mammalian cells could for instance include genes that confer resistance to drugs, such as neomycin, hygromycin, and methotrexate. The choice of selectable markers is within the level of ordinary skill in the art. Selectable markers can be introduced into a mammalian cell together with the gene of interest, or introduced, incorporated on the same plasmid. This type of constructs are known in the art (for example, Levinson and Simonsen, U.S. Pat. No. 4,713,339).

Transfected mammalian cells are allowed to grow for a period of time, typically a few days, to begin expressing the DNA sequence(s) introduced, Drug selection is then applied to select for growth of cells that are expressing the selectable marker in a stable fashion. For cells that have been transfected with an amplifiable selectable marker the drug concentration may be increased stepwise to select in order to increase the copy number of the cloned sequences, thereby increasing expression levels.

Methods thought to be useful for introducing expression vectors encoding gla-domainless prothrombin, is not used in this present invention due to the fact that, in the present invention encompasses the methodology in which thrombin will be activated during purification by proteases such as factor Xa.

### Culturing Conditions

Mammalian cells are generally cultured in commercially available serum-containing or serum-free media. Selection of a medium appropriate for the particular cell line used is within the level of ordinary skill in the art.

Variants of the commercially available HEK 293T cells lines and their suitable growth—and expression media may be used to further improve protein production yields. Variants of commercially available expression vectors including different promoters, secretion signals, transcription enhancers, etc., may also be used to improve protein production yields.

The scope of this invention encompasses the use of human cell lines as for instance immortalized human cells or even immortalized kidney cell lines such as human embryonic kidney (HEK) 293T cell line, or variations hereof, as well as stem cells or precursor cells.

Any cultured human cells may be used as host cells within the present invention. Preferred cultured mammalian cells for use include the COS-1 (ATCC CRL 1650), BHK, and 293 (ATCC CRL 1573; Graham et al., J. Gen. Virol. 6:59-72 (1977)) cell lines. A preferred BHK cell line is the BHK 570 cell line (deposited with the American Type Culture Collection under accession number CRL 10314). However, we have found that a human cell line selected seems to secrete significantly better outcome with more authentic-like human proteins and/or peptides; other human cell lines may have these capabilities.

### Method for the Preparation of Recombinant Human Thrombin

Methods are disclosed for producing thrombin. The protein is produced from host cells transformed or transfected with DNA construct(s) containing information necessary to direct the expression of thrombin precursors. The DNA constructs generally include the following operably linked elements: a transcriptional promoter, DNA sequence encoding a prothrombin, and a transcriptional terminator, however, as an integrate part of this invention a gla-domain containing prothrombin or rather, a recombinant human gla-domain containing prothrombin. Thrombin precursors produced from transformed or transfected host cells are activated either in vivo or in vitro. There is therefore a need in the art for methods for producing thrombin that is essentially free of contaminating proteins. The present invention fulfils this need and provides other related advantages.

It is within the scope of this invention that you may start up with a transfected monolayer culture of HEK 293 or HEK 293T, but it is within the scope of this invention that first, the cDNA for the particular protein, in this case the gla-domain containing prothrombin, is transfected into E. coli, and when one has increased the amount of DNA necessary to transfect the HEK 293 or HEK 293T cells, this is done using a monolayer culture of these cells, followed by an adaptation of these cells to a suspension cell culture, —transferred at the same time to an appropriate serum-free medium, after having performed the appropriate cloning and the cells are then cultured and optimized using the serum free medium. The cell culture can then either be harvested batch wise every 7-8 days or even more or less, when the measurement of the supernatant reveals a satisfactory content of the protein to be harvested and purified, followed by the appropriate activation of the gla-domain containing prothrombin to thrombin using a method developed by HumanZyme (HumanZyme, Inc., Chicago).

In regards to the human kidney cell and human retina-derived PER-C6 cells or more specifically, the human embryonic kidney (HEK) 293T cell line—containing DNA constructs of the present invention encoding a prothrombin that will be activated during the purification of proteases such as factor Xa.

For all the types of proteins to be obtained from human immortalized cell lines encompassed in this invention are cDNA copies of certain human variants of recombinant prothrombin.

Selection of optimal media is within the level of the skill in the art. Certain thrombin precursors may preferably be produced from these transfectants by the addition of heparin or thrombin. The activation of thrombin precursors containing a thrombin cleavage site in place of the wild-type thrombin activation site (Arg-Ile) may be enhanced by heparin added to the medium. Preferably, in this scenario, between 0.5 and 5.0 U/ml of heparin is added to the serum-free medium, more preferably between 1 and 5 U/ml and most preferably 1 U/ml of heparin is added to the serum-free medium according to some authors. To activate the protein produced from human cells, more specifically human kidney cell and human retina-derived PER-C6 cells, or more specifically human embryonic kidney cell and other cell lines (e.g., HEK 293, HEK 293T, HEK 293S, HEK 293 EBNA), stem cells, precursor cells or alike, containing DNA constructs of the present invention encoding a prothrombin, or a gla-domain containing prothrombin, wherein the thrombin will be activated during the purification by proteases such as factor Xa or Va, a thrombin activation site, which will have an anticipated activity between 0.5 and 5 .mu.g/ml of thrombin, which when added to the serum-free medium, more preferably will have an anticipated activity between 1 and 2 mu.g/ml of thrombin, with 1 mu.g/ml of thrombin added to the serum-free medium as being particularly preferred.

Thrombin precursors may be purified by conventional chromatography, and the thrombin precursor may then be activated by for instance snake venom activator in a serial dilution related to the protein concentration. Alternatively, thrombin precursors may be purified by affinity chromatography using anti-thrombin antibodies. Other methods of thrombin purification have been suggested as for instance in U.S. Pat. No. 4,965,203. According to the present invention the recombinant human thrombin will be activated during purification by proteases such as factor Xa.

Purified thrombin precursors may also be activated using the proteolytical activation of prothrombin as described by Heldebrant et al. and others (Heldebrant C M, Butkowski R J, Bajaj S P, Mann K G. J. Biol. Chem. (1973) 248:7149-7163; Downing M R, Butkowski R J, Clark M M, Mann K G, J. Biol. Chem. (1975), 250:8897; Krishnaswamy S, Church W R, Nesheim M E, Mann K G. J. Biol. Chem. (1987) 262:3291). Thrombin precursors that contain a thrombin activation site may be activated by the addition of thrombin. The activated thrombin can then be purified using column chromatography with a salt gradient. Methods of protein purification are well known in the art, for general purposes, see Scopes R. (Scopes, R., Protein Purification, Springer-Verlag, NY (1982), the higher purity that can be obtained the more clear-cut reaction of the thrombin, so a purification between 70-90% would most probably be ideal.

In the present invention the recombinant human thrombin is produced by activating said protein during purification by proteases such as factor Xa using the novel invention in obtaining the final product thrombin, to be used as a coagulant, to stabilized clots as for instance together with fibrinogen or as a carrier of cells for transplantation or implantation into mammals including humans in order to keep the cells transplanted in the target area or target organ, alternatively together with fibrinogen and/or collagen, especially collagens of the types I, II, III, IV, V, VI, VII, VIII, IX, X, XI, XII, XIII, XIV, XV, XVI, XVII, XVIII, XIX, or a mixture of the collagens, which again chemicals or drugs (e.g., diagnostics or medicinal drugs).

One embodiment according to the present invention provides a polynucleotide encoding a recombinant human thrombin precursor molecule as defined in SEQ ID NO: 17, the polynucleotide containing i) Gla domain as defined SEQ ID NO: 16. In another embodiment, the present invention further comprises ii) Kringle 2. In yet another embodiment, the present invention further comprises a polynucleotide as described herein, wherein the polynucleotide further comprises iii) HCP4 (protein C), and the recombinant human thrombin precursor molecule encoded has HPC4 linked at the 5′ end (SEQ ID NO: 6).

In an embodiment the recombinant human thrombin precursor molecule according to the present invention has autolytic activity.

One embodiment according to the present invention provides a vector comprising the polynucleotide as defined in the present invention. In a further embodiment the polynucleotide according to the present invention is further operably linked to control sequences recognised by a host cell transformed with said vector. In yet a further embodiment present invention the vector according the present invention is in the form of a plasmid vector.

One embodiment according to the present invention provides a human host cell comprising one or more vectors as defined in the present invention. In a further embodiment the human host cell is a human embryonic kidney (HEK) cell. In yet a further embodiment the human host cell is selected from the group consisting of HEK 293, HEK 293T, HEK 293S and HEK 293 EBNA.

One embodiment according to the present invention provides a method for the preparation of recombinant human prothrombin or thrombin using a human expression system. In a further embodiment the human expression system comprises a vector as defined herein. In yet a further embodiment the human expression system is a human host cell as defined herein. In yet a further embodiment the human expression system is cultured under serum-free conditions.

In an embodiment according to the present invention the recombinant human prothrombin or thrombin prepared according to the present invention contains a gla domain (SEQ ID NO: 16).

One embodiment according to the present invention provides the recombinant human prothrombin or thrombin has at least 85%, at least 90%, at least 95%, at least 96%, at least 97%, at least 98%, at least 99% or 100%, nucleic acid homology with the natural human prothrombin or thrombin gene.

One embodiment according to the present invention provides the recombinant human prothrombin or thrombin has at least 80%, at least 85%, at least 90%, at least 95%, at least 96%, at least 97%, at least 98%, at least 99% or 100%, amino acid homology with the natural human prothrombin or thrombin.

One embodiment according to the present invention provides at least 90%, such as at least 91%, at least 92%, least 93%, at least 94%, least 95%, at least 96%, least 97%, at least 98%, least 99%, or 100%, of the protein has a glycosylation pattern that results in an immunogenicity response substantially identical to that of the natural human prothrbmbin or thrombin.

In an embodiment according to the present invention the recombinant human thrombin clots fibrinogen at a faster rate than the natural human thrombin.

In an embodiment according to the present invention the recombinant human thrombin retains at least 50%, such as at least 60%, at least 70%, at least 80%, at least 90%, or 100%, of the initial fibrinogen polymerization activity after one week of storage at 4-8° C.

In an embodiment according to the present invention the recombinant human prethrombin or prothrombin can be activated by any means that are known to a person skilled in the art. In a preferred embodiment of the present invention the recombinant human prethrombin or prothrombin is activated to recombinant human thrombin by use of ecarin. Activation with ecarin has the further advantages of making immobilisation of the activator possible; ecarin therefore provides for a one step purification method of recombinant human thrombin.

In an embodiment according to the present invention the recombinant human prothrombin has a significantly lower autolytic activity than human natural prothrombin.

In an embodiment according to the present invention the human recombinant prothrombin is as defined in SEQ ID NO: 2 or SEQ ID NO: 4. In a further embodiment the human recombinant prothrombin as defined herein is for use in medicine.

In an embodiment according to the present invention the human recombinant prethrombin is as defined in SEQ ID NO: 7 or SEQ ID NO: 4. In a further embodiment the human recombinant prothrombin as defined herein is for use in medicine.

In an embodiment according to the present invention the human recombinant thrombin is as defined in SEQ ID NO: 17. In a further embodiment the human recombinant thrombin as defined herein is for use in medicine.

### Method for the Preparation of Recombinant Human Fibrinogen

Regarding the production of human recombinant fibrinogen, which approaches the authenticity of natural human fibrinogen, human cells or human kidney cell and human retina-derived PER-C6 cells lines, or more specifically the human embryonic kidney (HEK) 293T cell line may be the same type(s) of cells, which has been transfected with cDNA copies of human fibrinogen claimed in this invention, the cells propagated in serum-free medium and producing the human fibrinogen to approach similarity to authentic human fibrinogen from the cells due to the incorporation of the human genes encoding the α, β, and γ chains in the kidney cells secreting the more homogenous fibrinogen in serum-free medium with better functionality and with more consistent lot to lot reproducibility.

An embodiment according to the present invention provides a polynucleotide encoding recombinant human alpha fibrinogen corresponding to SEQ ID NO: 10 (the amino acid sequence in SEQ ID NO: 11) expressed in vitro in a human cell.

An embodiment according to the present invention provides a polynucleotide encoding recombinant human beta fibrinogen corresponding to SEQ ID NO: 12 (the amino acid sequence in SEQ ID NO: 13) expressed in vitro in a human cell.

An embodiment according to the present invention provides a polynucleotide encoding recombinant human gamma fibrinogen corresponding to SEQ ID NO: 14 (the amino acid sequence in SEQ ID NO: 15) expressed in vitro in a human cell.

One embodiment according to the present invention provides a vector comprising one or more of the polynucleotides as defined herein. In a further embodiment the vector comprises all three polynucleotides as defined herein. In yet a further embodiment the vector according to the present invention comprises the polynucleotide operably linked to control sequences recognised by a host cell transformed with said vector. In yet a further embodiment the vector according to the present invention is in the form of a plasmid vector.

One embodiment according to the present invention provides a human host cell comprising one or more vectors as defined herein. In a further embodiment the human host cell is a human embryonic kidney (HEK) cell. In yet a further embodiment the human host cell is selected from the group consisting of HEK 293, HEK 293T, HEK 293S and HEK 293 EBNA.

One embodiment according to the present invention provides a method for the preparation of recombinant human fibrinogen using a human expression system. In a further embodiment the human expression system comprises a vector as defined herein. In yet a further embodiment the human expression system is a human host cell as defined herein. In yet a further embodiment the human expression system is cultured under serum-free conditions.

One embodiment according to the present invention provides recombinant human fibrinogen has at least 85%, at least 90%, at least 95%, at least 96%, at least 97%, at least 98%, at least 99% or 100%, nucleic acid homology with the natural human fibrinogen gene.

One embodiment according to the present invention provides recombinant human fibrinogen has at least 80%, at least 85%, at least 90%, at least 95%, at least 96%, at least 97%, at least 98%, at least 99% or 100%, amino acid homology with the natural human fibrinogen.

One embodiment according to the present invention provides at least 90%, such as at least 91%, at least 92%, least 93%, at least 94%, least 95%, at least 96%, least 97%, at least 98%, least 99%, or 100%, of the protein has a glycosylation pattern that results in an immunogenicity response substantially identical to that of the natural human fibrinogen.

One embodiment according to the present invention provides a human recombinant fibrinogen comprising alpha chain as defined in SEQ ID NO: 11, beta chain as defined in SEQ ID NO: 13 and gamma chain as defined in SEQ ID NO: 15 and expressed in vitro in a human cell. In a further embodiment according to the present invention a human recombinant fibrinogen according to claim 43 is for use in medicine.

### Method for the Preparation of Recombinant Human Collagen

It is contemplated that the present method also may be used to produce other recombinant human proteins such as recombinant human collagen. Accordingly, in the following is given a brief description in this respect.

The collagen will be co-expressed with prolyl 4-hydroxylase, which hydroxylates specific proline residues of collagen, which has been transfected with cDNA copies of human collagen such as for instance any of the 19-20 known human collagens such as Collagen types I, II, III, IV, V, VI, VII, VIII, IX, X, XI, XII, XIII, XIV, XV, XVI, XVII, XVIII, XIX, —or any of these known collagens. In the absence of praline hydroxylation, the essential triple-helical conformation of collagen is thermally unstable for instance below physiological temperature.

These human collagen types, propagated in cells propagated in serum-free medium and producing the human collagen(s) or, more specifically, collagen of any of the types of the following collagens, e.g., I, II, III, IV, V, VI, VII, VIII, IX, X, XI, XII, XIII, XIV, XV, XVI, XVII, XVIII, XIX, in serum-free medium, to approach similarity to authentic human collagen(s) and/or specifically collagen type III. In this invention we have preferred to use human cells (among these stem cells, precursor cells or alike) for all these proteins, and more specifically, human kidney cell and human retina-derived PER-C6 cell lines, and even more specifically, a human embryonic kidney (HEK) 293T cell line, which is an immortalized cell line, described by P. Chen et al. in 2002 (Chen, P., et al., Protein Expression and Purification (2002) 24:481-488).

The following collagens may be found in humans and theoretically used in the production of CFM or CFM-Cell: Collagen types I, II, III, IV, V, VI, VII, VIII, IX, X, XI, XII, XIII, XIV, XV, XVI, XVII, XVIII, XIX, —all of these known collagens. Then there are a few recently detected collagens such as collagen type XX: recently collagen subfamily; type XXI: Type XXI collagen is an extracellular matrix component of the blood vessel walls, secreted by smooth-muscle cells; XXIII: recently found collagen in metastatic tumors; XXIV a fibrillar vertebrate collagen; XXVI: new member of collagens found in ovary and testes; XXVII: novel highly conserved collagen (pro(alpha)1(XXVII), type XXIII: recently found collagen in metastatic tumors; XXIV a fibrillar vertebrate collagen; XXVI: new member of collagens found in ovary and testes; XXVII: novel highly conserved collagen (pro(alpha)1(XXVII). Any of the above mentioned collagens may be used in form of human collagens or human recombinant collagens, or parts of human recombinant collagens, also called “synthetic human natural collagens”. The human recombinant collagen types described in this invention such as for instance, collagen type I, collagen II and/or collagen III, but not limited to these three types, —may be added to create an optimal tissue sealant. The recombinant human collagen prepared according to this invention in human cells, and more specifically human kidney cell and human retina-derived PER-C6 cells or even more specifically, cell lines called HEK 293, HEK 293T, HEK 293S, and HEK 293 EBNA. Stem cells or precursor cells may also be used in this regard. The resulting secreted collagen will be prepared in an appropriate reagent such as e.g. in a medium capable of dissolving the collagen (there are over 20 known collagens, whereof human derived or human recombinant derived collagens will be preferred); examples of suitable media include diluted hydrochloric acid (e.g. 10 mM) or other diluted inorganic or organic acid or an aqueous medium having a pH of 7 or less.

### Applications of Human Recombinant Proteins According to the Present Invention

Recombinant thrombin and a human recombinant collagen based matrix named HUMA-SEALANTT™-C, encompassed in this invention is a biological tissue sealant, which enables the usage of human recombinant thrombin produced by over-expression of the protein in specialized human cells, either together with human recombinant fibrinogen produced by over-expression of the protein in specialized human cells. Furthermore, human recombinant collagen or collagens such as for instance any of the approximately 20 human known types.

An optimal tissue sealant may also be accomplished by utilizing the human recombinant thrombin and the human recombinant fibrinogen, which even may be controlled in regards to the gelation of the tissue sealant by adding a recombinant aprotinin, and even this recombinant aprotinin may be produced by human cells or even by human kidney cell and human retina-derived PER-C6 cells. It may be an advantage to utilize multipotent cells from human umbilical cord or human umbilical cord blood to obtain a cell source that may be even more optimal in producing said proteins, obtaining a solid adherence, producing an easy and safe product as biological tissue sealant, to be used e.g. for wound healing, diffuse bleeding from organs and for any sealant purpose where fibrin glue or known tissue sealant normally containing either natural human proteins or in many cases, the addition of bovine (or other animal derived) proteins, which in relation to human natural non recombinant proteins may be capable of transmitting microbial (including viral) organisms or even, besides this risk, may be able to produce immunogenic reactions, due to for instance xenogeneic proteins (animal proteins as seen in tissue sealant products such as Tisseel® or Beriplast®, where the proteins are of natural human origin and not of recombinant origin, or in the case of tissue sealant such as both Tiseel and Beriplast, aprotinin is of bovine origin. The above described combinations or parts thereof may be used as vehicle for and guidance of cells during cell transplantation, and as carrier(s) of diagnostic or therapeutic drugs.

The present invention relates to a novel concept and method for preparing one or more compositions of human recombinant proteins that either is used in combinations as for instance recombinant human thrombin(s) and recombinant human thrombin mutants with high pro-coagulating activity and low anti-coagulating activity, and with cell signalling capability equal to or around the same signalling capability as wild type human thrombin and, in certain conditions with low or almost no existing degree of autolysis, rendering these capable of retaining at least their pro-coagulating enzymatic activity among others to human fibrinogen and recombinant human fibrinogen even when kept at refrigerator temperature (ranging from 3 to 17° C.) rendering these relatively stable recombinant thrombin useable in hemostatic kits kept at refrigerated temperature for an extended period such as for instance weeks or months. These recombinant human thrombin and human recombinant thrombin mutants shall be capable of reacting with recombinant human fibrinogen(s) to create hemostatic effect, clotting effect, gelating effect and adhering effect at least around equal to or better than other recombinant or natural thrombin to create fibrin. The recombinant human thrombin and fibrinogen shall be non-toxic to mammal cells including human cells, and capable of being injected together with cells or tissue, and in that way be capable of acting as cell carrier or capable of having adhesive effect together with cells for said cells to be injected, or implanted to target areas thus aiding cells to work as repair cells or substitute cells when implanted in the organism or in particular organs or tissue. The fibrin produced by the recombinant thrombin and recombinant fibrinogen or with at least one of these components being recombinant and the other being natural (e.g., fibrinogen).

The recombinant fibrin will be capable of substituting all other products, where natural fibrin-, non-toxic to live cells, is used, but with the advantage that it solely consists of recombinant human proteins resembling authentic human proteins, and mutants thereof in order to specifically obtain for instance recombinant-human thrombin(s) mutants especially highly capable of cleaving fibrinogen(s) thereof recombinant mammal, including recombinant human fibrinogen(s) and at the same time capable of showing PAR 1 signalling activity, and at the same time showing little or no activity on protein C.

Said recombinant fibrin will have the advantage of not carrying any microbial or infective material that otherwise relatively frequently is found in natural fibrin, derived from natural (human or bovine or from any other species) thrombin that has been reacting with natural fibrinogen or human or mammal. It has relatively frequently been noted, that natural thrombin and natural fibrinogen from blood of mammal origin may carry unnecessary risks of infecting humans or mammals with microbials (e.g., hepatitis B, hepatitis C, virus transferred from other species, such as for instance bovine, etc.), and even pathological proteins such as prions from humans or mammals including those of bovine origin.

Apart from being a cell carrier, which is a minor niche for the product made by using recombinant proteins within the scope of this invention, this particular tissue sealant (contrary to tissue sealants such as for instance Tisseel) has a broad area of application during cell transplantation surgery.

It is also within the scope of this invention to combine recombinant collagens or more specifically, recombinant human collagens, e.g., collagen type I, and/or type 2, and/or type 3 with recombinant or natural fibrinogen as described in U.S. provisional patent No. 60/722,366, in which application it has been shown that recombinant human collagen (e.g., type III) and form a gelating and adhering (clotting) effect together with fibrinogen.

It is within the scope of this invention to produce recombinant human fibrinogen such as fibrinogen made in mammal cells containing the cDNA for all three Aα, Bβ and γ genes that are expressed to create the protein. It is also within the scope of the invention that recombinant fibrinogen and/or recombinant thrombin can be combined with recombinant mammal such as recombinant human collagens (e.g., recombinant collagen type I or III) as described above.

Another approach within the scope of this invention is the usage of one single recombinant human protein such as for instance certain recombinant Human thrombin analogue(s) and/or recombinant thrombin mutant(s), produced in mammal cells intended to be either used individually as sole product(s) without other combinations for local hemostasis or in certain bleeding areas in the organism where the applied recombinant human thrombin can react with fibrinogen present in the bleeding area, and thus prevent or stop the bleeding. It is also within the scope of this invention to produce recombinant human fibrinogen such as fibrinogen made in mammal cells containing all three Aα, Bβ and γ genes that are expressed to create the protein where this product can be used as one single product. This recombinant human fibrinogen having a HMW at around 330,000 to 340,000 daltons for the treatment of genetic disorders such as hypofibrinogenimia or dysfibrogenimic disorders, and especially as part of the treatment of disseminated intravascular coagulation (DIC). The recombinant human fibrinogen product, when used as an individually administered biologics, it will be formulated in such a manner that it can be administered by injection/infusion in mammals including humans.

In particular, it is also within the scope of this invention to focus on creating said sole proteins that may mimic human active proteins participating in repairing bleeding disorders such as for instance disseminated coagulation disorders, or more inherent disorders, where patients may be lacking or having dys-functioning proteins that otherwise would participate in appropriate coagulation, as for instance disorders with impaired inherited fibrinogen deficiency syndroms or other abnormalities in the creation of intact fibrinogen proteins, such as dysfibrinogenesis, hypofibrinogenesis disorders or especially in disorders such as intravascular disseminated coagulation (DIC).

The Unique Recombinant Tissue Sealant Made from the Same Type of Human Cell Lines to Obtain Authentic Natural Proteins Constituting the Tissue Sealant

In order to obtain a unique tissue sealant, the procedure in producing the recombinant human proteins must approach proteins that appears to be next to or virtually the same as authentic natural human proteins. In other inventions the usage of CHO cells have been described as producers of recombinant thrombin, but we claim that one will not obtain a protein approaching a uniquely authentic protein optimally without using a technology that utilize the cDNA copies transfected into human cells or especially into human kidney cell and human retina-derived PER-C6 cells, or even more specifically into human embryonic kidney (HEK) 293T cell line.

In one embodiment the concentration of thrombin according to the present invention, such as recombinant human natural thrombin or recombinant human thrombin with one or more point mutations, may be less than 20 NIH units/ml, such as 1-20 NIH units/ml, such as less than 15 NIH units/ml, less than 10 NIH units/ml, less than 5 NIH units/ml, or less than 1 NIH unit/ml.

In one embodiment the concentration of fibrinogen according to the present invention, such as recombinant human natural fibrinogen, may be less than around 50 mg/ml, such as 1-50 mg/ml, such as less than 40 mg/ml, less than 30 mg/ml, less than 20 mg/ml, less than 10 mg/ml, or less than 2 mg/ml.

If the recombinant fibrinogen obtained furthermore, is produced using a quite different technology that make the other component of the tissue sealant kit different due to the fact that it may not live up to the requirement set forth in this invention by being produced in yet another way that may distance the protein in the kit, in regards to its authenticity to a human natural protein, the tissue sealant would thus not at all be as unique as the tissue sealant kits claimed in this invention. For instance in U.S. Pat. No. 6,780,411 a combination of recombinant fibrinogen, described as being produced as a recombinant fibrinogen being prepared by a process which involve the production of the recombinant fibrinogen in body fluids of transgenic mammals, such as sheep, pigs, cattle, goats, rabbits and camels. Such process is described in WO-A-9523868, incorporated in the above patent (U.S. Pat. No. 6,780,411). The other protein involved in the tissue sealant in this invention is recombinant thrombin which is described as being produced by mammal cells such as CHO cells. This combination is therefore significant different from the tissue sealants claimed in our invention from the point of view of the significant different concept in the production of the proteins constituting one of the tissue sealants described by us from the point of view that one protein (thrombin) is made in animal cells (CHO cells) and the other protein (fibrinogen) is made in body fluids of transgenic animals.

Recombinant human thrombin has a wide range of uses ranging from the treatment of coagulation disorders in humans to act as enzymatic initiator of clotting, treatment of burns, of skin grafting, both in minor and in major surgery either alone or as part of a product, such as tissue sealants.

The formulation of various wound tissue adhesives is discussed in detail in U.S. Pat. Nos. 4,427,650, 4,442,655, and 4,655,211, each of which is incorporated herein by reference.

The effective doses of recombinant human thrombin will vary considerably ranging from 100 to 15,000 units depending on the manner in which thrombin is used (the specific activity of pure thrombin is 3,000 units per μg protein).

The recombinant human thrombin made according to this invention may extremely small amounts that normally would not initiate a significant clotting, but when used with collagen of any type, such as for instance recombinant human collagens such as types I, II, III, IV, V, VI, VII, VIII, IX, X, XI, XII, XIII, XIV, XV, XVI, XVII, XVIII, XIX. Therefore, it is anticipated that—contrary to other inventions encompassing the usage of thrombin for creating gelating as well as adhering compounds—where thrombin are used in the area of 100 units to 15,000 units—the amount of thrombin used to induce a gelating (clotting) and/or adhering substance may indeed according to this invention, when used together with collagen and fibrinogen, or with collagen atone, may be significantly lower than people well experienced in the art would have anticipated as possible, and is therefore considered unique for this invention—namely the use of trace of recombinant natural or recombinant thrombin up to 90 units of thrombin added, in order to activate a gelation (clotting) and/or adherence, may indeed be sufficient to obtain said gelation (clotting).

Quite another model of this invention could be the use of some or one of the proteins which are within the scope of this invention that conveniently could be incorporated in substances which again could react with substances added to provoke gelation, adherence or gluing effect, as well as hemostatic effects.

Recombinant human prothrombin or thrombin according to the present invention may be formulated with any known pharmaceutically acceptable excipients.

Recombinant human fibrinogen according to the present invention may be formulated with any known pharmaceutically acceptable excipients.

The present invention additionally encompasses various kits which contain the ingredients and instruments required to carry out and implement the methods herein described, including use as a tissue sealant and/or a hemostatic agent. In one aspect, such a kit may include one or more substances selected from the group consisting of the recombinant human thrombins as recited herein, human fibrinogens as recited collagen, such as collagen of the types I, II, and III and such as recombinant human collagen, protease, such as serine protease, e.g. aprotinin, and one or more pharmaceutically acceptable excipients for use in medicine.

Additional compositions, kits, ingredient assemblies are included within the scope of the present invention for use in connection with the methods herein described as the follows.

### Items

1. A composition including the following proteins expressed in human cell lines transfected with the individual plasmid constructs containing the cDNA, which individually expresses the following proteins
a. recombinant human prothrombin
b. recombinant human thrombin
b. recombinant human fibrinogen
c. recombinant human collagen
2. Any of the recombinant human collagens of the types I, II, III, IV, V, VI, VII, VIII, IX, X, XI, XII, XIII, XIV, XV, XVI, XVII, XVIII, XIX comprising the recombinant human collagen described in item 1c.
3. A composition of a mixture of any of the human recombinant proteins described in items 1 and 2, constituting a gel-forming consistency.
4. A composition of a mixture of recombinant human collagen and recombinant human thrombin used in non-physiological extremely small amounts such as a number of units of thrombin from trace of thrombin to 90 units of thrombin will create a gel-forming consistency with or without the addition of fibrinogen.
5. A composition of a mixture of any of the proteins itemed in items 1-2, which, when applied to a surface, which may be biological of origin, which in vitro and in vivo adheres to said surface.
6. Composition of the above human recombinant proteins referred to under 1a-1c comprising human recombinant proteins using cDNA transfection in human stem cells and/or in further differentiated human precursor cells that will secrete one or more of the proteins in item 1, which, when brought into combinations with each other, will form a gel-like substance.
7. Compositions of the above human recombinant proteins referred to under 1a-1c comprising human recombinant proteins prepared using cDNA transfection in HEK 293, HEK 293T, HEK 293S, HEK 293 EBNA cell lines that will secrete one or more of the proteins in item 1, which, when brought into combinations with each other, will form a gel-like substance
8. Compositions of the above human recombinant proteins referred to under 1a-1c comprising human recombinant proteins prepared using cDNA transfection in HEK 293, HEK 293T, HEK 293S, HEK 293 EBNA cell lines that will secrete one or more of the proteins in item 1, which, when brought into combinations with each other, will form a gel-like substance that will adhere to surfaces such as biological surfaces in vitro and in vivo.
9. Any composition according to items 1 to 5, which under said conditions can be mixed with live cells and be used as carrier of said cells.
10. Any composition according to items 1 to 5, which under said conditions can be mixed with chemical compounds, acting as carrier for said chemical compounds in vivo for diagnostic and/or for therapeutic application.
11. A composition according to any of the preceding items for use in carrying medicinal drugs to target tissue or target organs.
12. Recombinant mammal thrombin with high pro-coagulant activity and low protein C (anti-coagulant) activating capability expressed in mammal cells
13. Recombinant mammal fibrinogen produced in mammal cells with cDNA expressing, with six polypeptide chains, 2 Aα, 2 Bβ, 2γ capable of functioning as a mammal plasma derived fully functioning mammal fibrinogen.
14. A product consisting of a kit containing recombinant mammal or human thrombin as described in item 12 for use in mammals.
15. A product consisting of a kit containing recombinant mammal or human fibrinogen as itemed in item 13 for use in mammals.
16. A product consisting of a kit containing recombinant mammal or human thrombin as itemed in item 12 and containing recombinant human fibrinogen as described in item 13, which is capable of creating fibrin.
17. A product consisting of a kit containing recombinant mammal fibrinogen as itemed in item 13 and recombinant mammal collagen, of collagen types I, II, and/or III for use as a sealant and a dermafilling product.
18. A recombinant mammal thrombin as itemed in item 12 and a recombinant mammal collagen of types I, II, and III for use as a sealant.
19. A method as defined herein, wherein the cells are cultured for a period of 5 to 20 days in suspension culture, and the cells are separated from the supernatant which contains the proteins.
20. A method as defined herein, wherein the recombinant human fibrinogen is purified by one or more methods selected from the group of anion exchange chromatography, affinity chromatography, affinity chromatography with protamine-agarose, immunoprecipitation with monoclonal or polyclonal antibody, affinity chromatography with monoclonal antibody, precipitation with ammonium sulphate.
21. A method according one or more of the above-recited items, wherein the fibrinogen is purified in the presence of at least one protease inhibitor.

An overview of the sequence listings enclosed is shown in Table 1 below.

TABLE 1

| SEQ ID | |
|---|---|
| NO | Description |
| 1 | Polynucleotide sequence of BC051332 (Human Prothrombin) - |
| | M400A (Base# 1198, 1199, 1200; ATG → gcc) |
| 2 | Amino acid sequence of BC051332 (Human Prothrombin) - |
| | M400A (Base# 1198, 1199, 1200; ATG → gcc) |
| 3 | Polynucleotide sequence of BC051332 (Human Prothrombin) - |
| | wt |
| 4 | Amino acid sequence of BC051332 (Human Prothrombin) - wt |
| 5 | Polynucleotide sequence of BC051332 (Human Prethrombin)- |
| | M256A (Base# 766, 767, 768; ATG → gcc) |
| 6 | Polynucleotide sequence of BC051332 (Human Prethrombin)- |
| | M256A (Base# 766, 767, 768; ATG → gcc) with HPC4 |
| 7 | Amino acid sequence of BC051332 (Human Prethrombin)- |
| | M256A (Base# 766, 767, 768; ATG → gcc) with HPC4 |
| 8 | Polynucleotide sequence of BC051332 (Human Prethrombin)- |
| | wt |
| 9 | Amino acid sequence of BC051332 (Human Prethrombin)-wt |
| 10 | Polynucleotide sequence of Fibrinogen a (NM_021871) |
| 11 | Amino acid sequence of Fibrinogen a (NM_021871) |
| 12 | Polynucleotide sequence of Fibrinogen b (BC106760) |
| 13 | Amino acid sequence of Fibrinogen b (BC106760) |
| 14 | Polynucleotide sequence of Fibrinogen r (BC021674) |
| 15 | Amino acid sequence of Fibrinogen r (BC021674) |
| 16 | GLA domain (where X is defined as gamma-carboxyglutamic |
| | acid (Gla)). |
| 17 | Sequence of thrombin analogue M84A from Seq. (calculated |
| | from prothrombin Seq. no. 285) comprising alpha-thrombin |
| | light (A) chain and heavy (B) chain |

### LEGENDS OF FIGURES

FIG. 1 show the Prepro Prothrombin Structure (Degen & Davie (1987) Biochemistry 26: 6165-6177)

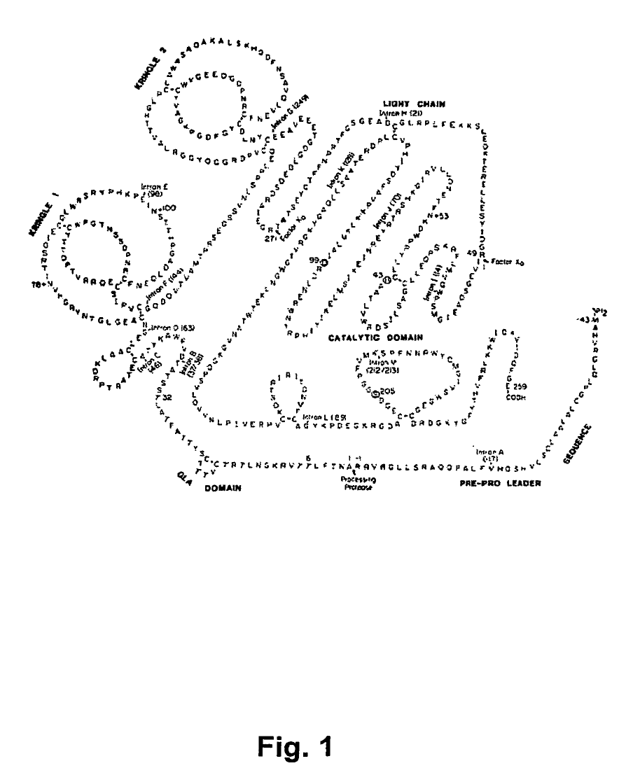

FIG. 2 is an overview of the structure of prethrombin M256A including the domains HPC4, Kringle 2, and Peptidase S. In addition, the site of the point mutation M256A is indicated.

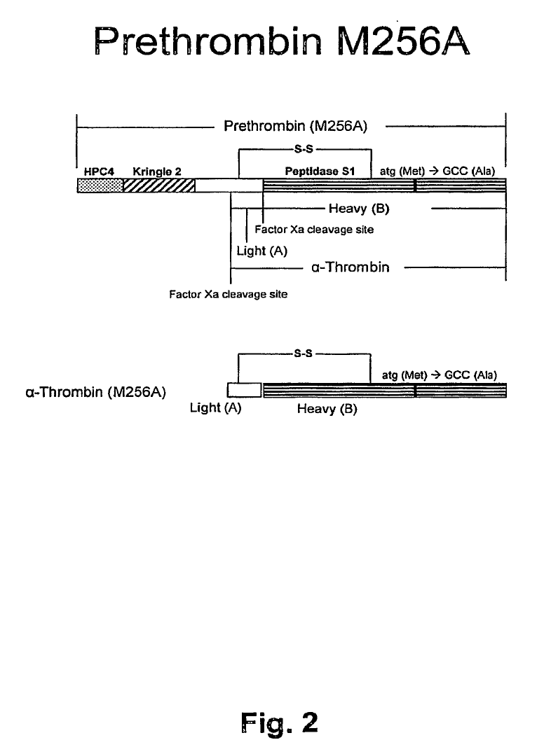

FIG. 3 is an overview of the structure of prothrombin M400A including the domains Gla, Kringle 1, Kringle 2, and Peptidase S. In addition, the site of the point mutation M256A is indicated.

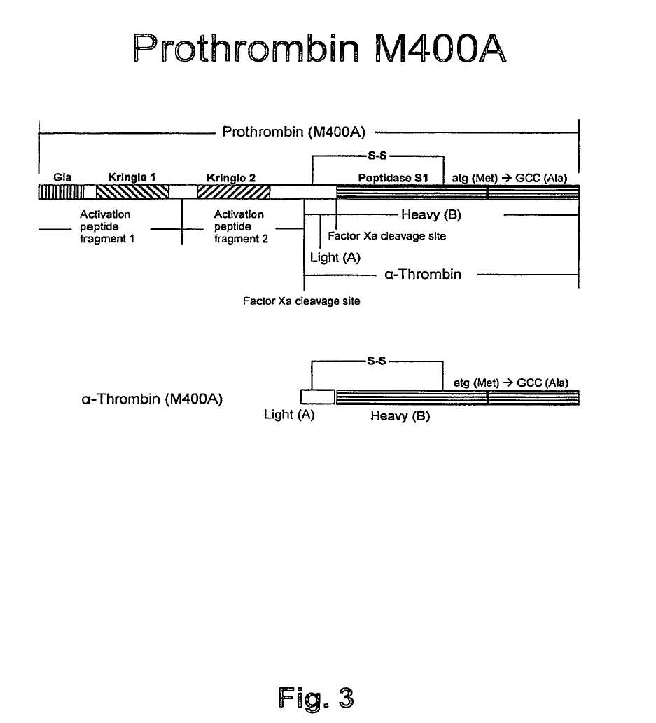

FIG. 4. In order to easily discriminate between the individual expressions such as prothrombins (either gla-domain (containing) prothrombin, gla-domainless prothrombin as used by Zymogenetics for their production of recombinant human thrombin, prethrombin, and prethrombin-1, FIG. 4 provides an overview over these individual terms. As illustrated in FIG. 4, prethrombin=prothrombin without gla domain and without kringle I domain (Gla-less domainless prothrombin). To facilitate purification HPC4 epitope was added on N-terminal. So, the first part of FIG. 4 shows the prothrombin including the Gla domain, and the Kringle 1 and Kringle 2. Furthermore, FIG. 4 illustrates the method of preparing recombinant human prethrombin starting with non-mutated prothrombin.

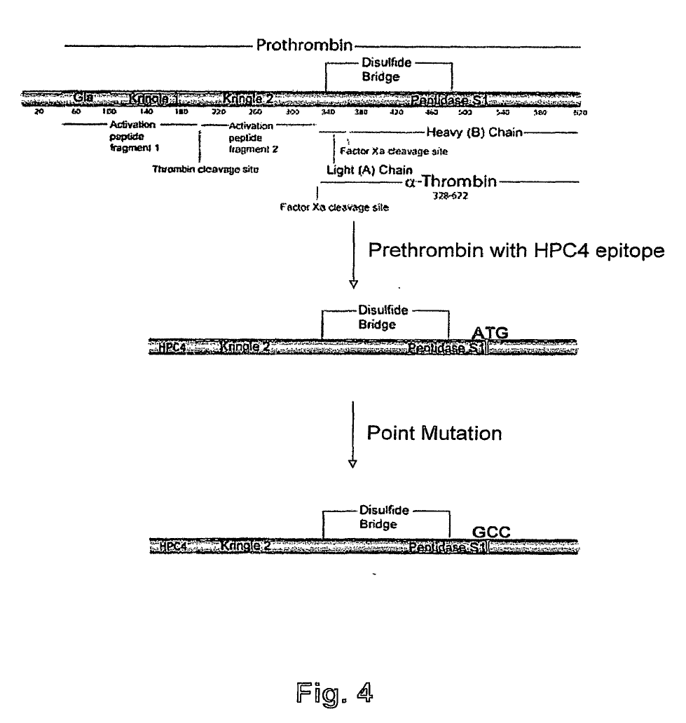

FIG. 5. The Gia domain of human prothrombin. GLA6 denotes y-carboxyglutamic acid residues, which when substituted by aspartic acid have little impact on prothrombin function; GLA7, GLA14, GLA19, GLA25, and GLA32 denote residues of intermediate importance to prothrombin function and GLA16, GLA26, and GLA29 denote those residues which are crucial to prothrombin function.

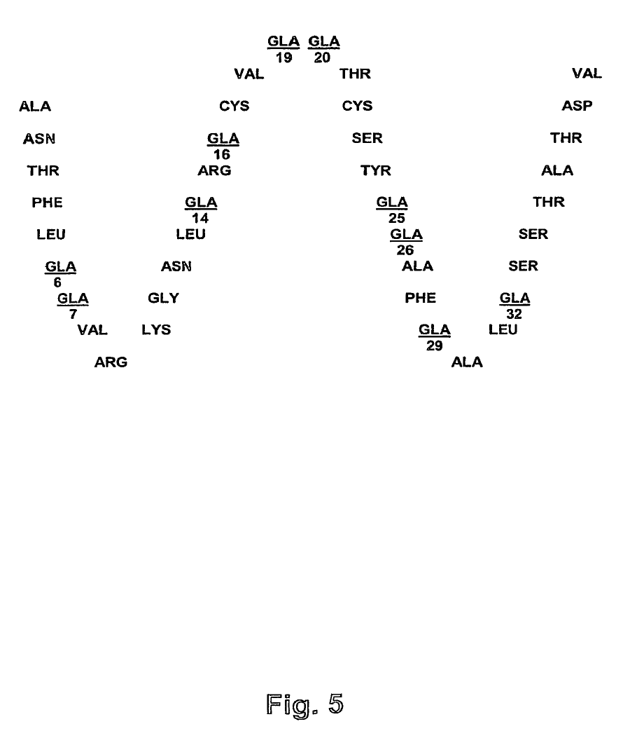

FIG. 6. Lane 1 is the molecular marker, Lane 2 is the control lane, Lane 3 is Prothrombin, Lane 4 is Prethrombin (wild type), Lane 5 is filtrate through MWCO of Lane # 4, Lane 6 is Prethrombin-1 M84A (Prethrombin analogue M256A), and Lane 7 is filtrate through 50 kD MWCO of Lane # 6.

FIG. 7. Untransfected Control cells, which died out (top 6 wells). The M84A (or Prethrombin analogue M256A) transfected cells showed continuous growth in the presence of antibiotics (antibiotic selection) are shown.

FIG. 8. The recombinant human thrombin analogue [M84A (or thrombin analogue M256A)] is shown. To the left a Coomassie blue staining of the medium containing 10% serum. To the right a Western blot reacted with monoclonal antibody against HPC4 (mAb-HPC4), where M84A (or analogue M256A) is visualized as a unique strong band, indicating a high concentration of M84A (or analogue M256A). This shows that when you have a relatively high amount of protein loaded (as seen in the Coomassie blue staining), a relatively high amount of contaminating proteins have to be removed during a purification of M84A (analogue M256A) from a serum containing medium.

FIG. 9. A Western blot is reacted with monoclonal anti HPC4 (mAb-HPC4) from serum-free suspension cell (HEK 293, adapted to suspension) culture.

FIG. 10 shows the Coomassie blue staining of serum-free protein yield, which is significantly more pure than samples containing 10% serum (left gel); the arrow in the left gel indicates the M84A location (or analogue M256A). The Western blot (right gel) is reacted with monoclonal antibody against HPC4 (mAb-HPC4) indicating a strong signal from M84A (or analogue M256A). It is observed that in lanes 2 and 3 on the left side, using Coomassie blue, the total amount of available proteins are visible. The amount of proteins observed is evaluated to be very small. The clones containing M84A (or analogue M256A) appears to be a relatively big part of the proteins (as shown using antibody detection of HPC 4) indicating that it will be very easy to purify the active protein from so relatively small amount of total protein,

FIG. 11 shows the purification of recombinant human thrombin analogue M84A (or analogue M256A). Lane 1 is the molecular marker. Lane 2 is the crude protein containing M84A, and in Lane 3 a completely purified M84A elute is observed.

FIG. 12. The recombinant human fibrinogen contained six polypeptide chains (a hexamer) (340 kDa), distributed as follows: 2Aa (66 kDa), 21313 (56-58 kDa), and 2γ (43-45 kDa).

FIG. 13. The gel was stained with simple blue staining. Lane 1 contained the Molecular Marker, Lane 2 fibrinogen, no heat and no DTT, Lane 3 Fibrinogen no heat no DTT, Lane 4 fibrinogen with heat and DTT, and Lane 5 fibrinogen with heat and DTT, meaning that Lane 3, 4 and 5 are under reducing conditions.

FIG. 14. Western Lane Order: Lane 1. MW marker, Lane 2. Fibrinogen, no heat, no DU, Lane 3. Fibrinogen with heat & DTT, Lane 4. Fibrinogen with heat & DTT, Lane 5. Fibrinogen with heat & DTT. Lane 3, 4 and 5 are under reducing conditions, under which the fibrinogen bands will disappear.

FIG. 15 shows Coomassie blue staining and Western blot analysis on the conditioned medium with Anti-Human prothrombin (Enzyme Research Laboratories). M: Marker, 1. Control medium; 2 prothrombin M84A (or Prothrombin Analogue M400A); 3 Prothrombin M84A (or Prothrombin Analogue M400A); 4 Prothrombin M84A (or Prothrombin Analogue M400A); 5 Prothrombin M84A (or Prothrombin Analogue M400A)

FIG. 16. Prethrombin M84A (or Prethrombin analogue M256A was observed using mAb Anti-Protein C (Roche).

FIG. 17. Prethrombin was eluted from crude Prethrombin from serum-free medium using Resin: anti-Protein C Affinity Matrix (Roche).

FIG. 18. Purification and activation of Prethrombin M84A (or Prethrombin analogue M256A to thrombin (α-thrombin M84A (or α-thrombin analogue M256A).

FIG. 19. A-thrombin Purification from heparin Sepharose Chromatography. Lane 1 shows the Ecarin activated α-thrombin mixture (or α-thrombin analogue M256A) was loaded on a heparin column pre-equilibrated with 10 mM Tris (pH 7.4)/200 mM Choline chloride. Lane 2 shows the Flow—thru which was collected until the baseline was obtained. Lane 3 shows Non-specific contaminant(s) which was washed with 10 mM Tris (pH 7.4)/500 mM Choline chloride. Lane 4 shows the α-thrombin M84A (or α-thrombin analogue M256A) eluted with 10 mM Tris (pH 7.4)/800 mM Choline chloride.

FIG. 20. Fibrinogen Expression from Serum-free suspension of the cells (suspension culture). S lane shows the human fibrinogen obtained in CHO cells (purified fibrinogen (1.4 μg) which were compared to Lane 1 and Lane 2. the human fibrinogen from HEK 293 (e.g., HEK 293T) cells from crude medium after day 6, (15 μl) in suspension culture in duplicate. Lanes S′, 1′ and 2′ show the fibrinogen split up into α, β, and γ after treatment with heat and DTT. Western blot reacted with poly rabbit anti-human fibrinogen (American Diagnostica Inc. #313R) were performed showing S and S′ (human fibrinogen (purified) from CHO cells before and after heat and DTT treatment, compared to 2 and 2′ recombinant human fibrinogen (crude) before and after heat and DTT treatment.

### EXAMPLES

### Example 1

### Method for the Preparation of Recombinant Human Prethrombin Via a Prethrombin with a HPC4 Domain

The human prothrombin gene was purchased from the Gene Bank (GB accession No. BC051332). PCR Gla-domain containing prothrombin region was obtained and an HPC4 (protein C) epitope was added to the 5′ end in the DNA. Protein C undergoes Ca²⁺-induced conformational changes required for activation by the thrombin-thrombomodulin complex. A Ca²⁺-dependent monoclonal antibody (HPC4) that blocks protein C activation was used to study conformational changes near the activation site in protein C as shown by Stearns et al (Stearns D J, Kurosawa S, Sims P J, Esmon N L, Esmon C T, The interaction of a Ca²⁺-dependent monoclonal antibody with the protein C activation peptide region. Evidence for obligatory Ca²⁺ binding to both antigen and antibody, J Biol Chem. (1988) 263(2):826-32).

Point mutation on 84 was made from Methionine to Alanine (ATG→GCC), see sequence of human M84A (SEQ ID NO: 6 and SEQ ID NO: 17), where the sequence (SEQ) numbering on B 116, corresponding to chymotrypsin “84” or prothrombin “400” (or prothrombin analogue M400A) was site mutated as described here, as a point mutation.

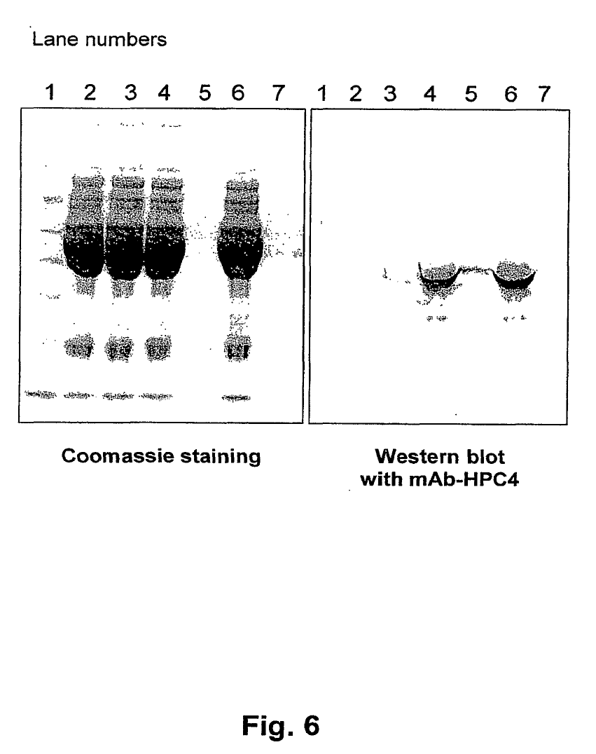

Another method for the production of recombinant human thrombin is to activate this thrombin from cloned recombinant Human prethrombin-1 as for instance M84A (or Prethrombin analogue M256A) was inserted into a pHZsec vector, which is a proprietary vector owned by HumanZyme Inc. (HumanZyme Inc., Chicago, USA). The M84A gene was then amplified in E. coli. The amplified gene was then transfected into human embryonic kidney cells (HEK 293) line in a monolayer cell culture in medium containing serum according to the method developed by HumanZyme Inc. (HumanZyme Inc., Chicago). The expression of the human recombinant thrombin analogue (M84A) was verified by Western blot analysis using mAb-HPC4. From FIG. 6 it is observed that both wild type prethrombin-1 and M84A prethrombin-1 have been successfully expressed from HEK 293 cells.

### Example 2

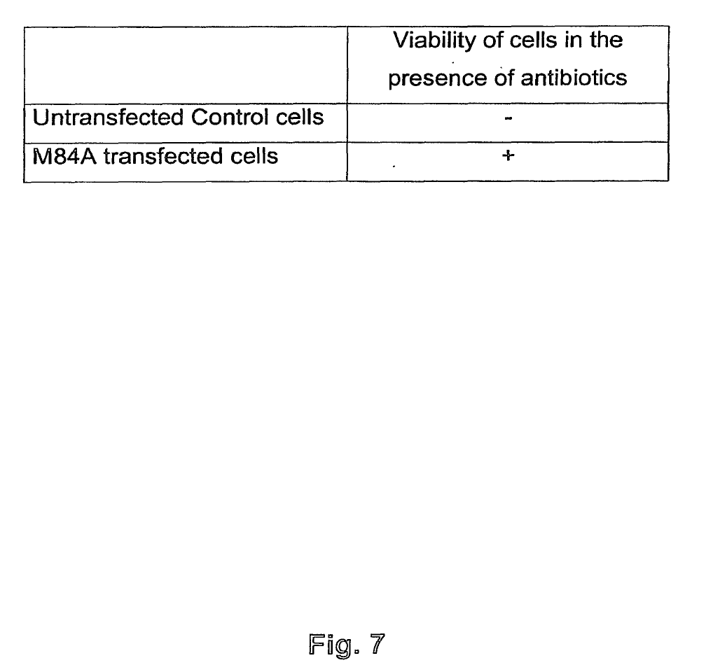

Recombinant human thrombin analogue (M84A (or analogue M256A) was expressed in HEK 293 cells. Twelve (12) clones were selected from 6 wells to larger plates. The two top clones based on Western blot analysis using monoclonal antibody against HPC4 (mAb-HPC4). The cells were adapted for serum free medium and processed as a suspension culture. The recombinant human prethrombin-1 M84A (or prethrombin analogue M256A) was purified using a pilot size purification method. The yield from a serum-free suspension culture of recombinant HU thrombin analogue (M84A (or thrombin analogue M256A)) per Liter was satisfactory. Untransfected cells (controls) died out while M84A (or analogue M256A) transfected cell lines showed continuous growth in the presence of antibiotics see FIG. 7.

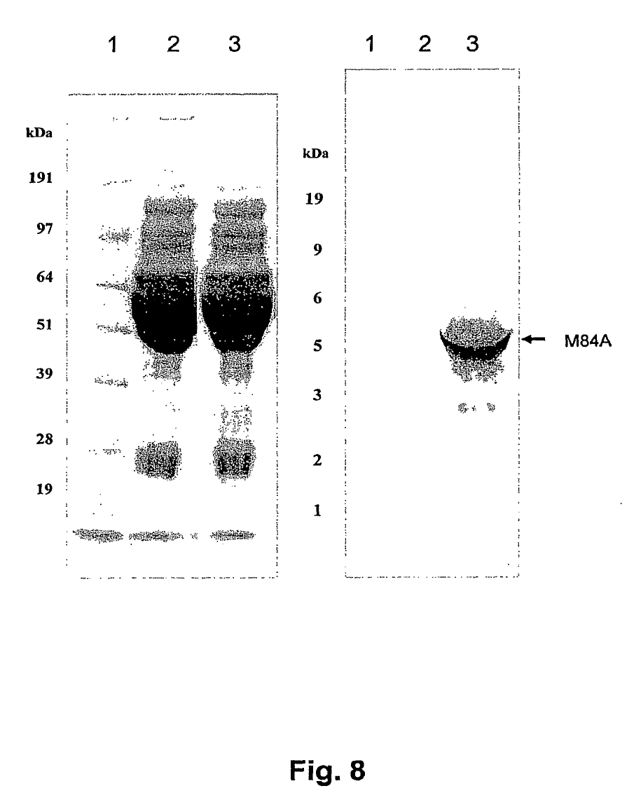

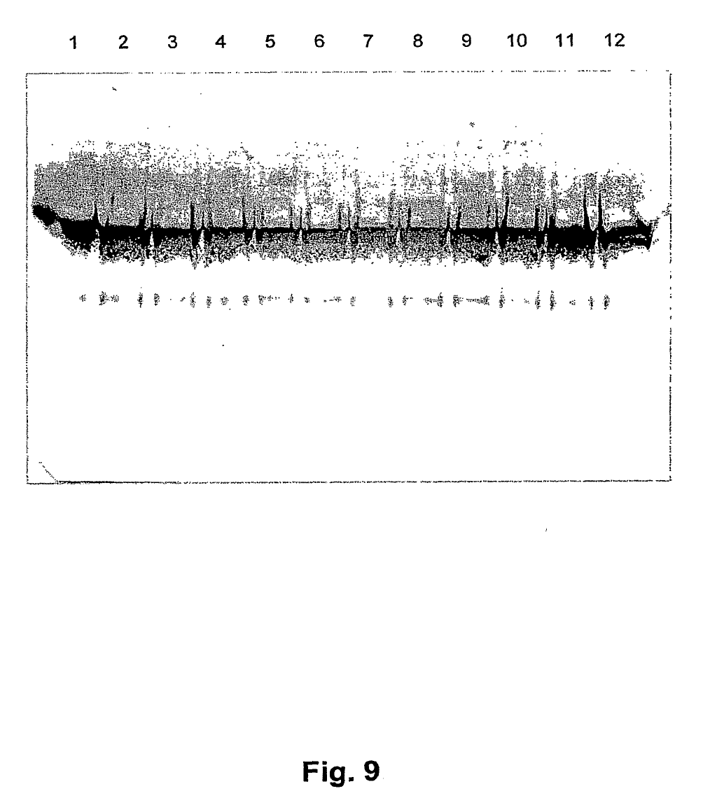

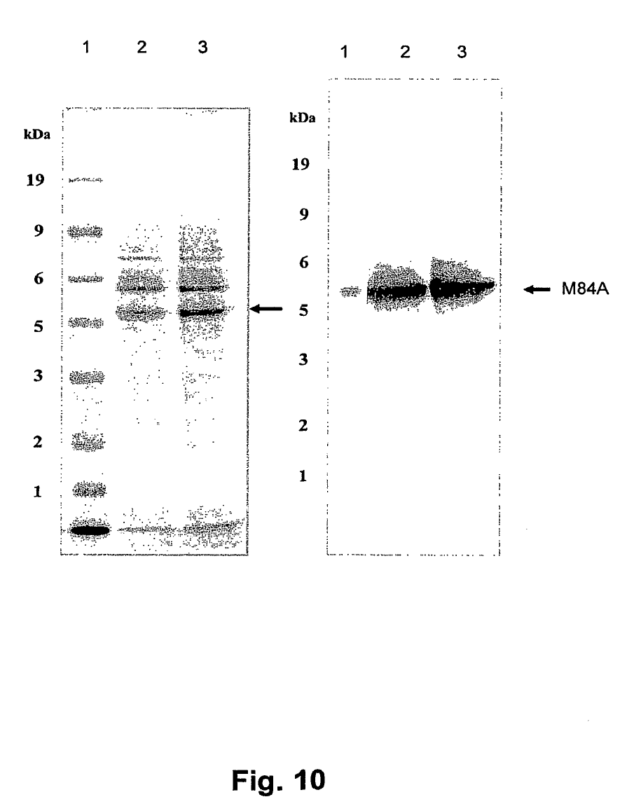

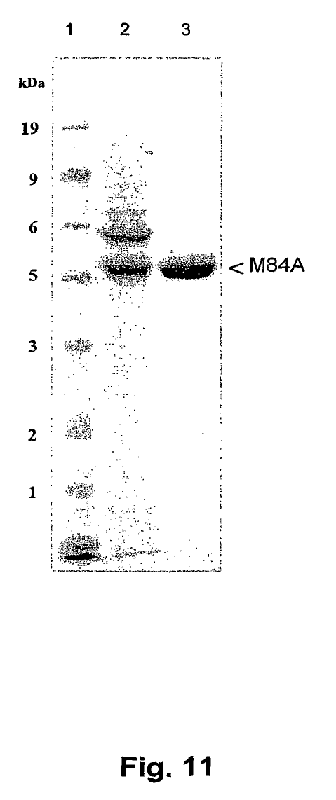

FIG. 8 shows the screening of 12 HEK 293 cell clones from which clones 2 and 11 were selected. FIG. 9 shows a Western blot reacted with monoclonal anti HPC4 (mAb-HPC4) from serum-free suspension cell (HEK 293, adapted to suspension) culture. FIG. 10 shows the two clones judged to give the top results in Western blot selected from FIG. 9. FIG. 11 shows the result of the purification of recombinant human prethrombin-1, M84A crude protein prior to purification, and eluted, purified M84A protein is shown in FIG. 10. FIG. 11 shows the purification of recombinant human thrombin analogue M84A. Lane 1 is the molecular marker. Lane 2 is the crude protein containing M84A (or prethrombin analogue M256A), and in Lane 3 a completely purified M84A (or prethrombin analogue M256A) elute is observed.

Insertion of the DNA sequence and the preparation, transfection, expression, purification, and activation are done by HumanZyme Inc. (HumanZyme Inc. Chicago, USA).

Amino acid sequence of human thrombin analogue M84A from Seq. (calculated from prothrombin Seq. no. 285) is according to SEQ ID NO 17.

This shows that it is relatively easy to purify recombinant human thrombin analogue M84A, when the crude protein prior to the elution only contains relatively few bands of proteins, when compared to the amount of proteins present in cell culture containing serum (as for instance 10% serum).

### Example 3

### Method for Preparing Prothrombin with Intact Gla Domain

Selection of stable cell line transfected with recombinant human gla-domain containing prothrombin, which can be activated to recombinant thrombin, recombinant human thrombins whereof one of the recombinant human thrombins is the recombinant human thrombin analogue, M84A or even recombinant wild type thrombin derived from activation of gla-domain containing prothrombin. The entire Gla-domain containing prothrombin gene is amplified in E. coli, and the amplified Gla-domain containing prothrombin is transfected into HEK 293 (e.g., HEK 293T) cells and grown in monolayer culture containing serum in the medium. Several clones were harvested and the clones indicating the content of M84A (or analogue M400A) was isolated and these clones were adapted into suspension cell culture in serum free medium. The M84A gla-domain prothrombin was harvested from the suspension cell culture by separating the cells from the supernatant. The M84A (or analogue M400A was then precipitated and purified and activated by one step method using a proteinase.

Amino acid sequence of human thrombin analogue M84A from Seq. (calculated from prothrombin Seq. no. 285) is according to SEQ ID NO: 17.

During the process, the recombinant Gla-domain prothrombin, a prothrombin containing signal peptide, propeptide, Gla domain, two kringle domains and protease domain (e.g., trypsin) is retained.

### Example 4

### Method for the Preparation of Fibrinogen

The recombinant human fibrinogen, and more specifically the recombinant human fibrinogen cloned in Human Embryonic Kidney cells (HEK 293), and even more specifically described as recombinant human authentic fibrinogen, due to the fact that the 6 polypeptides, 2 Aα, 2Bβ, and 2γ genes could most probably have been cloned into any suitable host cell, but in this invention were cloned into the HEK 293 cells. The human fibrinogen α, β, and γ genes was purchased as GB accession No.: NM_{—}021871 for Fb_Aα; BC106760 fpr Fb_Bβ and BC021674 for Fb_γ, and were tested using PCR for fibrinogen α,β, and γ genes. Each gene was cloned into a pHZsec vector. The genes were amplified in E. coli. The polypeptide sequence of human alpha (precursor) fibrinogen is according to SEQ ID NO: 10 and the amino acid sequence of human alpha (precursor) fibrinogen is according to SEQ ID NO: 11. The nucleic acid sequence of the beta chain of human fibrinogen is according to SEQ ID NO: 12 and the amino acid sequence of beta chain of fibrinogen is according to SEQ ID NO: 13. The polypeptide sequence of human gamma fibrinogen is according to SEQ ID NO: 14 and the amino acid sequence of human gamma fibrinogen is according to SEQ ID NO: 15.

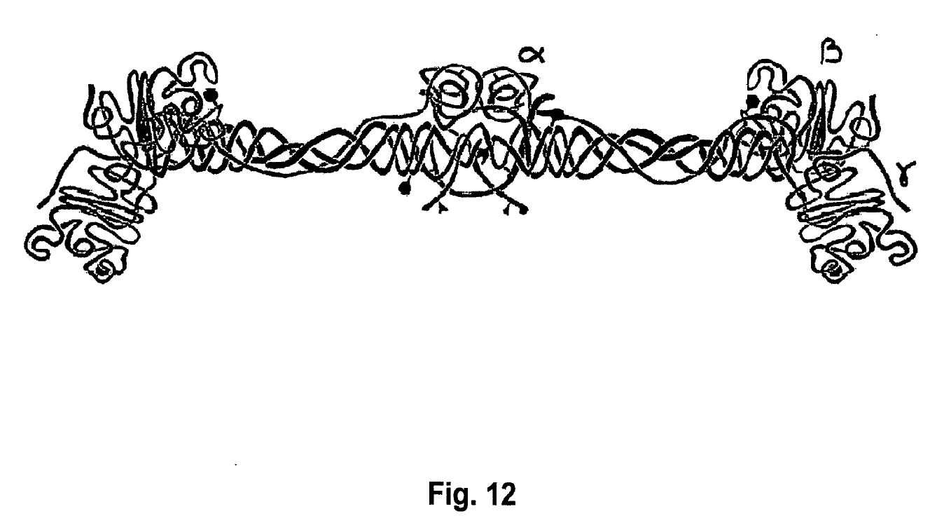

Thereafter the three genes were transfected into the HEK 293 cell line and grown in monolayer culture with serum. The expression was verified by Western blot analysis of the conditioned medium with poly Ab rabbit antihuman fibrinogen IgG. When comparing the structure of the recombinant fibrinogen, it appeared to be authentic when compared to natural human fibrinogen as seen in FIG. 12, which shows that the recombinant human fibrinogen contains six polypeptide chains (a hexamer) (340 kDa), distributed as follows: 2Aα (66 kDa), 2 Bβ (56-58 kDa), and 2γ(43-45 kDa).

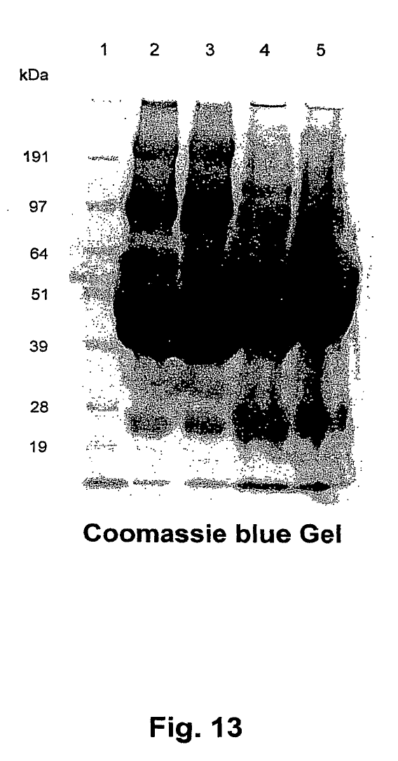

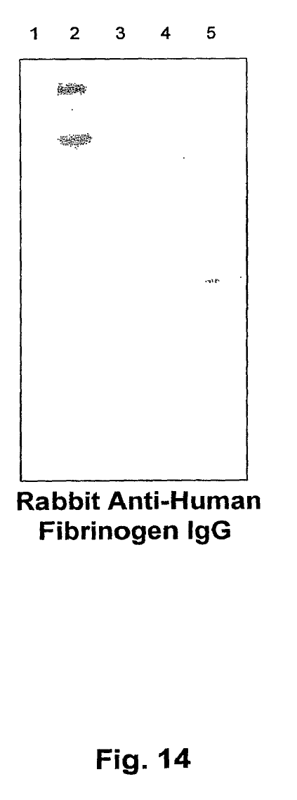

The expression of the recombinant (authentic) human fibrinogen in HEK 293 cell system is visualized in FIG. 13, where the gel is Coomassie blue stain gel, and in FIG. 14 the recombinant (authentic) human fibrinogen is visualized on Western blot using rabbit antihuman fibrinogen IgG.

The clones giving the highest yield was “antibiotic” selected and the yield of recombinant (authentic human fibrinogen was found to provide a satisfactory amount of recombinant human authentic fibrinogen from HEK 293 cells).

### Example 5

The human prothrombin gene was purchased from the Gene Bank (GB accession No. BC051332). PCR Gla-domain containing prothrombin region aa#44-622 (579 aa) was subjected to point mutation on 84 (Chy) (or at prothrombin analogue M400A) from Met to Ala (ATG→GCC)

Point mutation on amino acid position 84 (or at prothrombin analogue position 400) was made from methionine to alanine (ATG→GCC);

M84A-mF: 5′-GAAAAGATATCCGCCTTGGAAAAGATC-3′
M84a-mR: 5′-GATCTTTTCCAAGGCGGATATCTTTTC-3′

The recombinant Human Prothrombin M84A (or Human Prothrombin analogue M400A) was then inserted into the pHZsec vector, and amplification of the M84A (or analogue M400A) gene was done in E. coli. The amplified gene was then transfected into HEK 293 (e.g., HEK293T) cell line in monolayer in 10% serum containing medium. The expression was found and verified by Western blot analysis on the conditioned medium with Anti-Human prothrombin (Enzyme Research Laboratories).

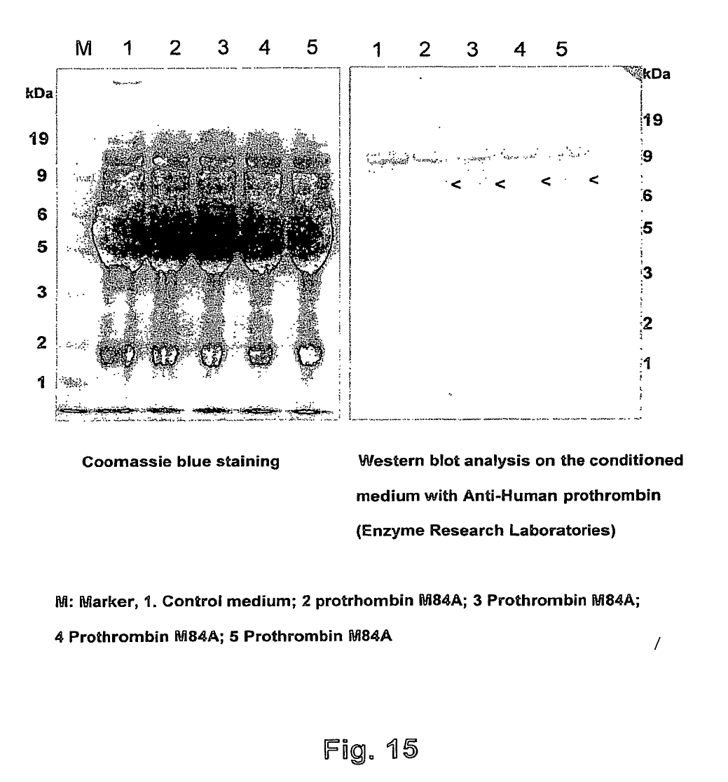

As can be seen on FIG. 15, the gla-domain Prothrombin M84A (or prothrombin analogue M400A) is expressed in Monolayer as evidenced by the Coomassie blue and Western analysis, which was performed from the medium supernatant from the cell culture. It appeared that all transfected HEK 293 cells in various wells expressed the prothrombin M84A (or prothrombin analogue M400A).

### Example 6

Human prothrombin gene was purchased from the Gene Bank (GB accession no. BC051332). PCR prethrombin region aa#206-622 (417 aa) and added HPC4 epitope (18 aa) at N-terminus (5′ end in DNA). Reference for HPC4 Stearns (Stearns D J, Kurosawa S, Sims P J, Esmon N L, Esmon C T. J. Biol. Chem. (1988), 263:826-832) as follows:

```
gcagcaaagcttgaagaccaagtagatccgcggctcattgatgggaaggt
  A A K L E D Q V D P R L I D G K V
cgacctgtca
    D L S
```

Point mutation M84A on amino acid position 84 (or at analogue position M256A) from Met to Ala (ATG→GCC) was done.

M84A-mF: 5′-GAAAAGATATCCGCCTTGGAAAAGATC-3′
M84a-mR: 5′-GATCTTTTCCAAGGCGGATATCTTTTC-3′

The cloned recombinant Human Thrombin M84A (or thrombin analogue M256A) was inserted into the pHZsec vector. The cloned gene was amplified in E. coli. The gene was transfected into HEK 293 (e.g., HEK 293T) cell line in monolayer culture. The expression was verified by Western analysis on the conditioned medium with mAb-HPC4 (see FIG. 8). Twelve clones were selected and cells from 6 wells were transferred to a larger plate. The top two clones based on Western analysis (mAB-FIPC4) were selected. These cells were adapted to serum-free medium.

### Pilot Purification of Prethrombin M84A (or Analogue M256A)

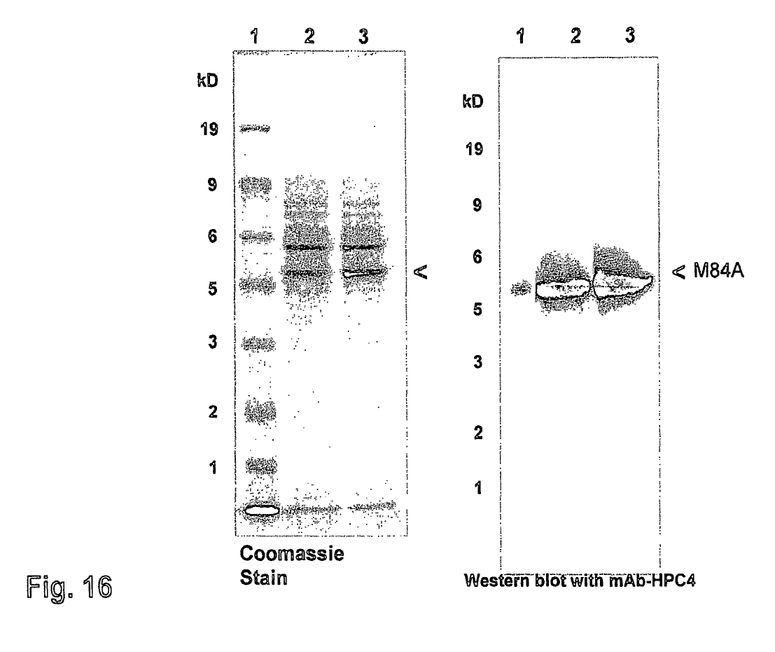

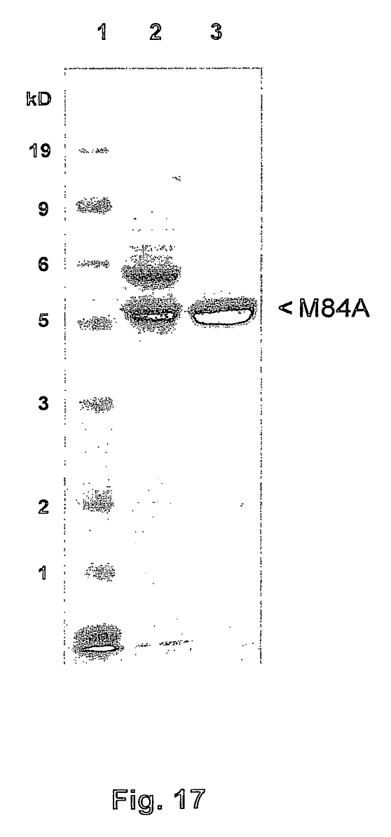

As it can be seen from FIG. 16, Prethrombin M84A (or analogue M256A was observed using mAb Anti-Protein C (Roche). In FIG. 17 it can be observed that Prethrombin is eluted from crude Prethrombin from serum-free medium using Resin: anti-Protein C Affinity Matrix (Roche).

### Thrombin M84A Activation by Ecarin

Ecarin was obtained from Sigma (Sigma E0504), and is Echis carinatus venom, it is independent of Ca++, phospholipids, and plasma clotting factors. It can be used as possible immobilization agent.

The activation reaction condition was as follows, 50 μl of Ecarin (50 EU/ml) was added to 5 ml of M84A prethrombin (or prethrombin analogue M256A) (0.5 mg/ml) either in crude media or in TBS (after purification through HPC4 column). Determination of the activation was done by using the thrombin assay with a chromogenic substrate (FPRpNA from Midwest Bio-Tech # 710013) at room temperature, and the following was done:
970 μl of 1×TBS (Tris-based saline, pH 7.4)
25 μl 1.7 mM FPRpNA (final concentration 40 μM)
5 μl reaction mixture at each time period.

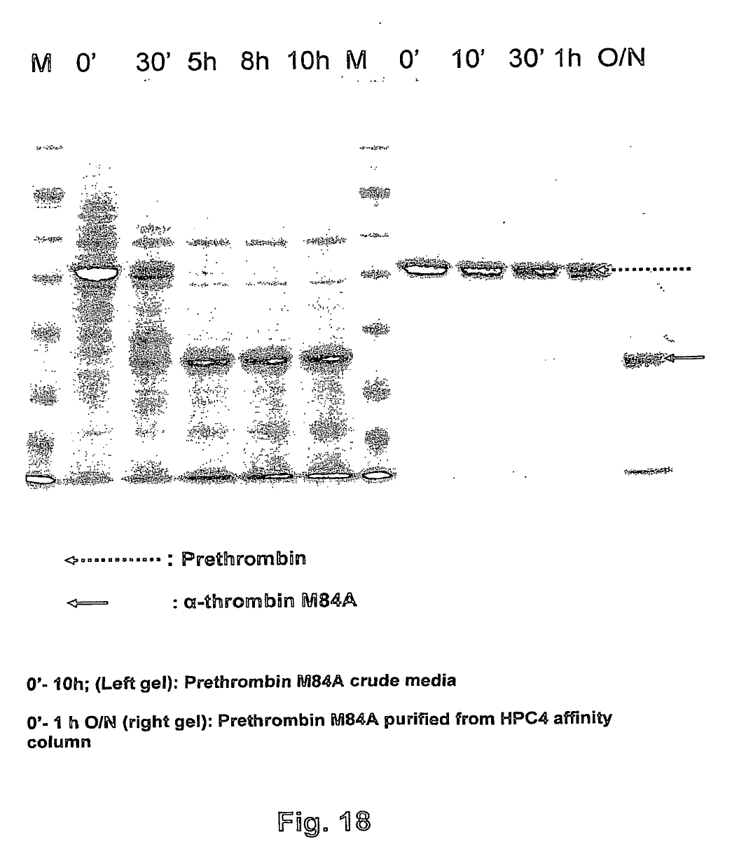

The purification and activation of Prethrombin M84A (or prethrombin analogue M256A) to thrombin (α-thrombin M84A) is shown in FIG. 18.

A-Thrombin Purification from Heparin Sepharose Chromatography

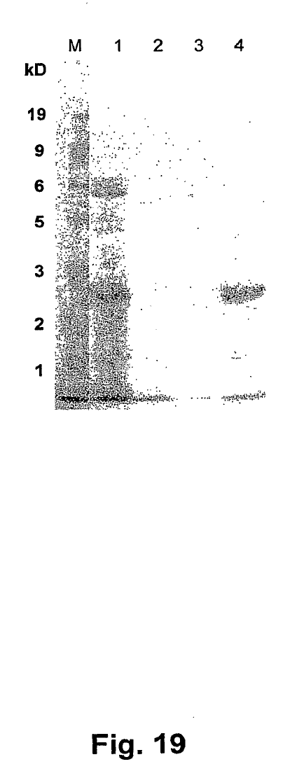

According to FIG. 19, Lane 1 shows the Ecarin activated α-thrombin mixture was loaded on a heparin column pre-equilibrated with 10 mM Tris (pH 7.4)/200 mM Choline chloride. Lane 2 shows the Flow—thru which was collected until the baseline was obtained. Lane 3 shows Non-specific contaminant(s) which was washed with 10 mM Tris (pH 7.4)/500 mM Choline chloride. Lane 4 shows the α-thrombin M84A eluted with 10 mM Iris (pH 7.4)/800 mM Choline chloride (see FIG. 19).

### Example 7

Human fibrinogen α, β, and γ genes were purchased from the Gene Bank (GB accession no: NM_{—}021871 for Fb_Aα, BC 106760 for Fb_Bβ, and BC021674 for Fb_γ. The PCR fibrinogen α, β, and γ genes were cloned, each gene into pHZec vector, and the genes were amplified in E. coli. The three genes were transfected into HEK 293 (e.g., HEK 293T) cell line in monolayer culture with 10% serum. The expression of fibrinogen was verified by Western blot analysis on the conditioned medium with poly AB rabbit anti-human fibrinogen IgG.

Fibrinogen Expression from Serum-Free Suspension of the Cells (Suspension Culture)

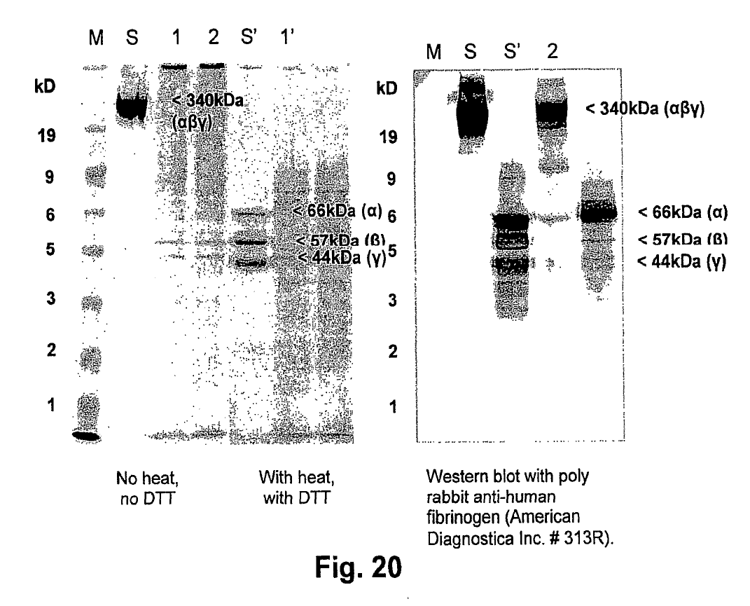

As shown in FIG. 20, S lane shows the human fibrinogen obtained in CHO cells (purified fibrinogen (1.4 μg), which were compared to Lane 1 and Lane 2, the human fibrinogen from HEK 293 (e.g., HEK 293T) cells from crude medium after day 6, (15 μl) in suspension culture in duplicate. Lanes S′, 1′ and 2′ show the fibrinogen split up into α, β, and γ after treatment with heat and DTT. Western blot reacted with poly rabbit anti-human fibrinogen (American Diagnostica Inc. #313R) were performed showing S and S′ (human fibrinogen (purified) from CHO cells before and after heat and DTT treatment, compared to 2 and 2′ recombinant human fibrinogen (crude) before and after heat and DTT treatment.

1-50. (canceled)

51. A method for preparing a recombinant human gla domain (SEQ ID NO: 16) containing prothrombin or thrombin comprising using a human expression system.

52. The method according to claim 51, wherein the human expression system comprises a human embryonic kidney (HEK) cell or a PER-C6 cell.

53. The method according to claim 52, wherein the HEK cell is selected from the group consisting of HEK 293, HEK 293T, HEK 293S and HEK 293 EBNA.

54. The method according to claim 51, wherein the human expression system is cultured under serum-free conditions.

55. The method according to claim 51, wherein the recombinant human prothrombin or thrombin has at least 85% of a nucleic acid sequence identity with the natural human prothrombin or thrombin gene.

56. The method according to claim 51, wherein the recombinant human prothrombin or thrombin has at least 80% of an amino acid sequence identity with the natural human prothrombin or thrombin.

57. The method according to claim 51, wherein at least 90% of the protein has a glycosylation pattern that results in an immunogenicity response substantially identical to that of the natural human prothrombin or thrombin.

58. The method according to claim 51, wherein the recombinant human thrombin clots fibrinogen more efficiently than the natural human thrombin.

59. A method according to claim 51, wherein the recombinant human thrombin retains at least 50% of the initial fibrinogen polymerization activity after one week of storage at 4-8° C.

60. A method according to claim 51, wherein the recombinant human prothrombin is activated to recombinant human thrombin by use of ecarin.

61. The method according to claim 51, wherein the human recombinant prothrombin is as defined in SEQ ID NO: 2 or SEQ ID NO: 4.

62. A recombinant human host cell comprising a vector encoding a gla domain containing human prothrombin or thrombin.

63. The recombinant human host cell of claim 62, which is a human embryonic kidney (HEK) cell or a PER-C6 cell.

64. The method according to claim 63, wherein the HEK cell is selected from the group consisting of HEK 293, HEK 293T, HEK 293S and HEK 293 EBNA.


SEQUENCE LISTING (17 sequences)

SEQ ID NO: 1 - DNA, 1743 bp, artificial
  Recombinant human thrombin precursor molecule
gccaacaccttcttggaggaggtgcgcaagggcaacctggagcgagagtgcgtggaggagacgtgcagctacgaggaggccttcgaggctctggagtcctccacggctacggatgtgttctgggccaagtacacagcttgtgagacagcgaggacgcctcgagataagcttgctgcatgtctggaaggtaactgtgctgagggtctgggtacgaactaccgagggcatgtgaacatcacccggtcaggcattgagtgccagctatggaggagtcgctacccacataagcctgaaatcaactccactacccatcctggggccgacctacaggagaatttctgccgcaaccccgacagcagcaccacgggaccctggtgctacactacagaccccaccgtgaggaggcaggaatgcagcatccctgtctgtggccaggatcaagtcactgtagcgatgactccacgctccgaaggctccagtgtgaatctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgcgtggtgctatgtggccgggaagcctggcgactttgggtactgcgacctcaactattgtgaggaggccgtggaggaggagacaggagatgggctggatgaggactcagacagggccatcgaagggcgtaccgccaccagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagtga

SEQ ID NO: 2 - protein, 579 aa, artificial
  Recombinant human thrombin precursor molecule
ANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGAWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISALEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 3 - DNA, 1743 bp, Homo sapiens; identical to SEQ ID NO: 1

SEQ ID NO: 4 - protein, 579 aa, homo sapiens
ANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGAWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 5 - DNA, 1257 bp, artificial
  recombinant human thrombin precursor molecule
ctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgcgtggtgctatgtggccgggaagcctggcgactttgggtactgcgacctcaactattgtgaggaggccgtggaggaggagacaggagatgggctggatgaggactcagacagggccatcgaagggcgtaccgccaccagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccgccttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagtga

SEQ ID NO: 6 - DNA, 1311 bp, artificial
  recombinant human thrombin precursor molecule
gcagcaaagcttgaagaccaagtagatccgcggctcattgatgggaaggtcgacctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgcgtggtgctatgtggccgggaagcctggcgactttgggtactgcgacctcaactattgtgaggaggccgtggaggaggagacaggagatgggctggatgaggactcagacagggccatcgaagggcgtaccgccaccagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccgccttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagtga

SEQ ID NO: 7 - protein, 435 aa, artificial
  human recombinant prethrombin
AAKLEDQVDPRLIDGKVDLSPPLEQCVPDRGQQYQGRLAVTTHGLPCLAWASAQAKALSKHQDFNSAVQLVENFCRNPDGDEEGAWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISALEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 8 - DNA, 1311 bp, artificial
  Human Prethrombin wild type with HPC4 protein
gcagcaaagcttgaagaccaagtagatccgcggctcattgatgggaaggtcgacctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgcgtggtgctatgtggccgggaagcctggcgactttgggtactgcgacctcaactattgtgaggaggccgtggaggaggagacaggagatgggctggatgaggactcagacagggccatcgaagggcgtaccgccaccagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagtga

SEQ ID NO: 9 - protein, 435 aa, artificial
  Human prethrombin wild type with HPC4 protein
AAKLEDQVDPRLIDGKVDLSPPLEQCVPDRGQQYQGRLAVTTHGLPCLAWASAQAKALSKHQDFNSAVQLVENFCRNPDGDEEGAWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 10 - DNA, 1911 bp, homo sapiens
gtcctaagtgtggtgggcacagcatggactgcagatagtggtgaaggtgactttctagctgaaggaggaggcgtgcgtggcccaagggttgtggaaagacatcaatctgcctgcaaagattcagactggcccttctgctctgatgaagactggaactacaaatgcccttctggctgcaggatgaaagggttgattgatgaagtcaatcaagattttacaaacagaataaataagctcaaaaattcactatttgaatatcagaagaacaataaggattctcattcgttgaccactaatataatggaaattttgagaggcgatttttcctcagccaataaccgtgataatacctacaaccgagtgtcagaggatctgagaagcagaattgaagtcctgaagcgcaaagtcatagaaaaagtacagcatatccagcttctgcaaaaaaatgttagagctcagttggttgatatgaaacgactggaggtggacattgatattaagatccgatcttgtcgagggtcatgcagtagggctttagctcgtgaagtagatctgaaggactatgaagatcagcagaagcaacttgaacaggtcattgccaaagacttacttccctctagagataggcaacacttaccactgatcaaaatgaaaccagttccagacttggttcccggaaattttaagagccagcttcagaaggtacccccagagtggaaggcattaacagacatgccgcagatgagaatggagttagagagacctggtggaaatgagattactcgaggaggctccacttcttatggaaccggatcagagacggaaagcccaaggaaccctagcagtgctggaagctggaactctgggagctctggacctggaagtactggaaaccgaaaccctgggagctctgggactggagggactgcaacctggaaacctggaagctctggacctggaagtactggaagctggaactctgggagctctggaactggaagtactggaaaccaaaaccctgggagccctagacctggtagtaccggaacctggaatcctggcagctctgaacgcggaagtgctggacactggacttctgagagctctgtatctggtagtactggacaatggcactctgaatctggaagttttaggccagatagcccaggctctgggaacgcgaggcctaacaacccagactggggcacatttgaagaggtgtcaggaaatgtaagtccagggacaaggagagagtaccacacagaaaaactggtcacttctaaaggagataaagagctcaggactggtaaagagaaggtcacctctggtagcacaaccaccacgcgtcgttcatgctctaaaaccgttactaagactgttattggtcctgatggtcacaaagaagttaccaaagaagtggtgacctccgaagatggttctgactgtcccgaggcaatggatttaggcacattgtctggcataggcaccctggatgggttccgccataggcaccctgatgaagctgccttcttcgacactgcctcaactggaaaaacattcccaggtttcttctcacctatgttaggagagtttgtcagtgagactgagtctaggggctcagaatctggcatcttcacaaatacaaaggaatccagttctcatcaccctgggatagctgaattcccttcccgtggtaaatcttcaagttacagcaaacaatttactagtagcacgagttacaacagaggagactccacatttgaaagcaagagctataaaatggcagatgaggccggaagtgaagccgatcatgaaggaacacatagcaccaagagaggccatgctaaatctcgccctgtcagaggtatccacacttctcctttggggaagccttccctgtccccctagtga

SEQ ID NO: 11 - protein, 625 aa, homo sapiens
ADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRGIHTSPLGKPSLSP

SEQ ID NO: 12 - DNA, 1389 bp, homo sapiens
caaggtgtcaacgacaatgaggagggtttcttcagtgcccgtggtcatcgaccccttgacaagaagagagaagaggctcccagcctgaggcctgccccaccgcccatcagtggaggtggctatcgggctcgtccagccaaagcagctgccactcaaaagaaagtagaaagaaaagcccctgatgctggaggctgtcttcacgctgacccagacctgggggtgttgtgtcctacaggatgtcagttgcaagaggctttgctacaacaggaaaggccaatcagaaatagtgttgatgagttaaataacaatgtggaagctgtttcccagacctcctcttcttcctttcagtacatgtatttgctgaaagacctgtggcaaaagaggcagaagcaagtaaaagataatgaaaatgtagtcaatgagtactcctcagaactggaaaagcaccaattatatatagatgagactgtgaatagcaatatcccaactaaccttcgtgtgcttcgttcaatcctggaaaacctgagaagcaaaatacaaaagttagaatctgatgtctcagctcaaatggaatattgtcgcaccccatgcactgtcagttgcaatattcctgtggtgtctggcaaagaatgtgaggaaattatcaggaaaggaggtgaaacatctgaaatgtatctcattcaacctgacagttctgtcaaaccgtatagagtatactgtgacatgaatacagaaaatggaggatggacagtgattcagaaccgtcaagacggtagtgttgactttggcaggaaatgggatccatataaacagggatttggaaatgttgcaaccaacacagatgggaagaattactgtggcctaccaggtgaatattggcttggaaatgataaaattagccagcttaccaggatgggacccacagaacttttgatagaaatggaggactggaaaggagacaaagtaaaggctcactatggaggattcactgtacagaatgaagccaacaaataccagatctcagtgaacaaatacagaggaacagccggtaatgccctcatggatggagcatctcagctgatgggagaaaacaggaccatgaccattcacaacggcatgttcttcagcacgtatgacagagacaatgacggctggttaacatcagatcccagaaaacagtgttctaaagaagacggtggtggatggtggtataatagatgtcatgcagccaatccaaacggcagatactactggggtggacagtacacctgggacatggcaaagcatggcacagatgatggtgtagtatggatgaattggaaggggtcatggtactcaatgaggaagatgagtatgaagatcaggcccttcttcccacagcaatagtga

SEQ ID NO: 13 - protein, 461 aa, homo sapiens
QGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 14 - DNA, 1272 bp, homo sapiens
gctcttttatttctctcttcaacatgtgtagcatatgttgctaccagagacaactgctgcatcttagatgaaagattcggtagttattgtccaactacctgtggcattgcagatttcctgtctacttatcaaaccaaagtagacaaggatctacagtctttggaagacatcttacatcaagttgaaaacaaaacatcagaagtcaaacagctgataaaagcaatccaactcacttataatcctgatgaatcatcaaaaccaaatatgatagacgctgctactttgaagtccaggaaaatgttagaagaaattatgaaatatgaagcatcgattttaacacatgactcaagtattcgatatttgcaggaaatatataattcaaataatcaaaagattgttaacctgaaagagaaggtagcccagcttgaagcacagtgccaggaaccttgcaaagacacggtgcaaatccatgatatcactgggaaagattgtcaagacattgccaataagggagctaaacagagcgggctttactttattaaacctctgaaagctaaccagcaattcttagtctactgtgaaatcgatgggtctggaaatggatggactgtgtttcagaagagacttgatggcagtgtagatttcaagaaaaactggattcaatataaagaaggatttggacatctgtctcctactggcacaacagaattttggctgggaaatgagaagattcatttgataagcacacagtctgccatcccatatgcattaagagtggaactggaagactggaatggcagaaccagtactgcagactatgccatgttcaaggtgggacctgaagctgacaagtaccgcctaacatatgcctacttcgctggtggggatgctggagatgcctttgatggctttgattttggcgatgatcctagtgacaagtttttcacatcccataatggcatgcagttcagtacctgggacaatgacaatgataagtttgaaggcaactgtgctgaacaggatggatctggttggtggatgaacaagtgtcacgctggccatctcaatggagtttattaccaaggtggcacttactcaaaagcatctactcctaatggttatgataatggcattatttgggccacttggaaaacccggtggtattccatgaagaaaaccactatgaagataatcccattcaacagactcacaattggagaaggacagcaacaccacctggggggagccaaacaggctggagacgtttaatga

SEQ ID NO: 15 - protein, 411 aa, homo sapiens
YVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 16 - protein, 39 aa, homo sapiens
  misc_feature (6)..(7): Xaa can be any naturally occurring amino acid
ANTFLXXVRKGNLXRXCVXXTCSYXXAFXALXSSTATDV

SEQ ID NO: 17 - protein, 295 aa, artificial
  Mutated Human alpha Thrombin M84A
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISALEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE